(19) United States
(12) Patent Application Publication
(10) Pub. No.: US 2003/0144187 A1
(43) Pub. Date: Jul. 31, 2003

(54) OPG FUSION PROTEIN COMPOSITIONS AND METHODS

(76) Inventors: COLIN R. DUNSTAN, THOUSAND OAKS, CA (US); SCOTT K.. WOODEN, THOUSAND OAKS, CA (US); MICHAEL B. MANN, THOUSAND OAKS, CA (US)

Correspondence Address: AMGEN INCORPORATED, MAIL STOP 27-4-A, ONE AMGEN CENTER DRIVE, THOUSAND OAKS, CA, 91320-1799, US

(21) Appl. No.: 09/389,782

(22) Filed: Sep. 3, 1999

(51) Int. Cl. A61K 38/17; C12P 21/02; C12N 5/06; C07H 21/04; C12N 9/64

(52) U.S. Cl. 514/12; 435/69.1; 435/320.1; 435/325; 435/226; 536/23.2

(57) ABSTRACT

The present invention relates to OPG fusion protein compositions, methods of preparation of such compositions and uses thereof. More particularly, the present invention relates to a fusion protein comprising an OPG polypeptide and an immunoglobulin FC region.

### FIELD OF THE INVENTION

The present invention relates to OPG fusion protein compositions and methods of preparation and use thereof.

### BACKGROUND OF THE INVENTION

The availability of recombinant proteins for therapeutic use has led to advances in protein modifications in order to enhance or improve the properties of such proteins as pharmaceutical agents. Such modifications can provide enhanced protein protection and decreased degradation by reducing or eliminating proteolysis. Additional advantages include, under certain circumstances, increasing the stability, circulation time, and the biological activity of the therapeutic protein. A review article describing protein modifications is Francis, Focus on Growth Factors 3:4-10 (May 1992) (published by Mediscript, London, UK).

One such modification is the use of an Fc region of an immunoglobulin molecule. Antibodies comprise two functionally independent parts, a variable domain known as “Fab”, which binds antigen, and a constant domain, known as “Fc” which provides the link to effector functions such as complement or phagocytic cells. The Fc portion of an immunoglobulin has a long plasma half-life, whereas the Fab is short-lived. (Capon, et al., Nature 337, 525-531 (1989)).

Therapeutic protein products have been constructed using the Fc domain to provide longer half-life or to incorporate functions such as Fc receptor binding, protein A binding, complement fixation and placental transfer which all reside in the Fc proteins of immunogobulins. Id. For example, the Fc region of an IgG1 antibody has been fused to the N-terminal end of CD30 ligand (CD30-L), a molecule which binds CD30 receptors expressed on Hodgkin's Disease tumor cells, anaplastic lymphoma cells, T-cell leukemia cells and other malignant cell types. See, U.S. Pat. No. 5,480,981. IL-10, an anti-inflammatory and antirejection agent has been fused to murine Fcγ2a in order to increase the cytokines short circulating half-life. (Zheng et al., The Journal of Immunology, 154, 5590-5600 (1995)). Studies have also evaluated the use of tumor necrosis factor receptor linked with the Fc protein of human IgG1 to treat patients with septic shock. (Fisher et al., N. Engl. J. Med., 334: 1697-1702 (1996); Van Zee et al., The Journal of Immunology, 156: 2221-2230 (1996)). Fc has also been fused with CD4 receptor to produce a therapeutic protein for treatment of AIDS. See, Capon et al., Nature, 337:525-531 (1989). In addition, the N-terminus of interleukin-2(IL-2) has also been fused to the Fc portion of IgG1 or IgG3 to overcome the short half life of IL-2 and its systemic toxicity. See, Harvill et al., Immunotechnology, 1, 95-105 (1995).

Osteoprotegerin (OPG) has been described in PCT Publication No. WO97/23614 and found to negatively regulate formation of osteoclasts in vitro and in vivo. OPG dramatically increased the bone density in transgenic mice expressing the OPG polypeptide and reduced the extent of bone loss when administered to ovariectomized rats. An analysis of OPG activity in in vitro osteoclast formation revealed that OPG blocks the differentiation of osteoclasts from monocyte/macrophage precursors. OPG appears to have specificity in regulating the extent of osteoclast formation. OPG is a potent factor in blocking bone resorption and may be used in the prevention and treatment of loss of bone mass. In vitro and in vivo activity of inhibiting osteoclast formation and blocking loss of bone was also observed in fusion proteins comprising OPG and an Fc domain.

Fusion of an OPG polypeptide to a heterologous protein or peptide such as an Fc domain may be carried out in a variety of different ways such that the resulting OPG fusion proteins may exhibit variable biological properties and potentially variable effectiveness as therapeutics. For example, an Fc domain may be fused either at the amino terminus or at the carboxy terminus of an OPG polypeptide, it may be fused directly or via a linking molecule, and/or one of the Fc or OPG moieties, or both, may be modified from their native forms. These different OPG fusion protein constructs may show variations in levels of expression, ease of isolation and/or purification, biological activity, and the like.

Consequently, there exists a need to develop OPG fusion protein compositions as effective therapeutics. Such compositions will exhibit advantageous properties relating to production, isolation, purification, biological activity, stability, and circulation time. The present invention provides such compositions.

### SUMMARY OF THE INVENTION

The invention provides for OPG fusion protein compositions, methods of preparation of such compositions and uses thereof and provided herein. More particularly, the present invention relates to an OPG fusion protein comprising an OPG protein, or variant, fragment, or derivative thereof, and an Fc protein, or variant, fragment or derivative thereof. Unexpectedly, it has been observed that fusion of an Fc region to a truncated OPG polypeptide demonstrates advantages which are not seen in unfused truncated or full-length OPG polypeptides. Such unexpected advantages contribute to lower doses and/or less frequent dosing of the polypeptides of the invention. Thus, as described below in more detail, the present invention has a number of aspects relating to the modification of polypeptides via fusion of an Fc region to an OPG protein (or variants, fragments or derivatives thereof), as well as, specific modifications, preparations and methods of use thereof.

In one aspect, the present invention provides for a protein having a formula selected from the group consisting of: R₁-R₂, R₂-R₁, R₁-L-R₂ and R₂-L-R₁ wherein R₁ is a Fc protein, or a variant or fragment thereof, R₂ is an OPG protein, or variant or fragment thereof, and L is a linker. The invention also provides for linkers of R₁ and R₂ moieties as described herein.

In another aspect, the present invention provides an OPG fusion protein wherein Fc (or a variant, fragment or derivative thereof) is genetically fused to the carboxy-terminus of an OPG protein (or a variant, fragment or derivative thereof). In another aspect of the invention, an Fc portion may also be linked to the carboxy-terminus of an OPG protein (or a variant, fragment or derivative thereof) by a peptide or chemical linker as known in the art. Additional aspects of the present invention include not only OPG fusion protein compositions, but also nucleic acid sequences encoding such proteins, related vectors and host cells containing such vectors, both useful for producing fusion proteins of the present invention.

In another aspect, the present invention provides for methods of preparing an OPG fusion protein. Using recombinant DNA methods available to one skilled in the art. Chemical methods for the synthesis and linking of OPG fusion polypeptides as also provided. Furthermore, such aspects include methods of protein production and purification as well.

In another aspect, the present invention provides methods for treating bone disorders, in particular, loss of bone mass. Such bone disorders include osteoporosis, lytic bone diseases resulting from tumor metastasis, hypercalcemia, Paget's disease, bone loss due to rheumatoid arthritis, and the like.

In another aspect, the present invention also provides for related pharmaceutical compositions of OPG fusion proteins, variants, fragments and derivatives thereof, for use in the above therapies.

### DESCRIPTION OF THE FIGURES

FIG. 1 shows the amino acid sequence of the hinge, CH2 and CH3 regions of human IgGγ1.

FIG. 2 shows the amino acid sequence of human OPG [1-401].

FIG. 3 shows the amino acid sequence of OPG[22-194]-Fc.

FIG. 4 shows the amino acid sequence of OPG[22-201]-Fc.

FIG. 5 shows the amino acid sequence of OPG[22-194]-FcΔC.

FIG. 6 shows the amino acid sequence of OPG[22-201]-FcΔC.

FIG. 7 shows the amino acid sequence of OPG[22-194]-FcG₁₀.

FIG. 8 shows the amino acid sequence of [met]FcΔC-OPG[22-194].

### DETAILED DESCRIPTION OF THE INVENTION

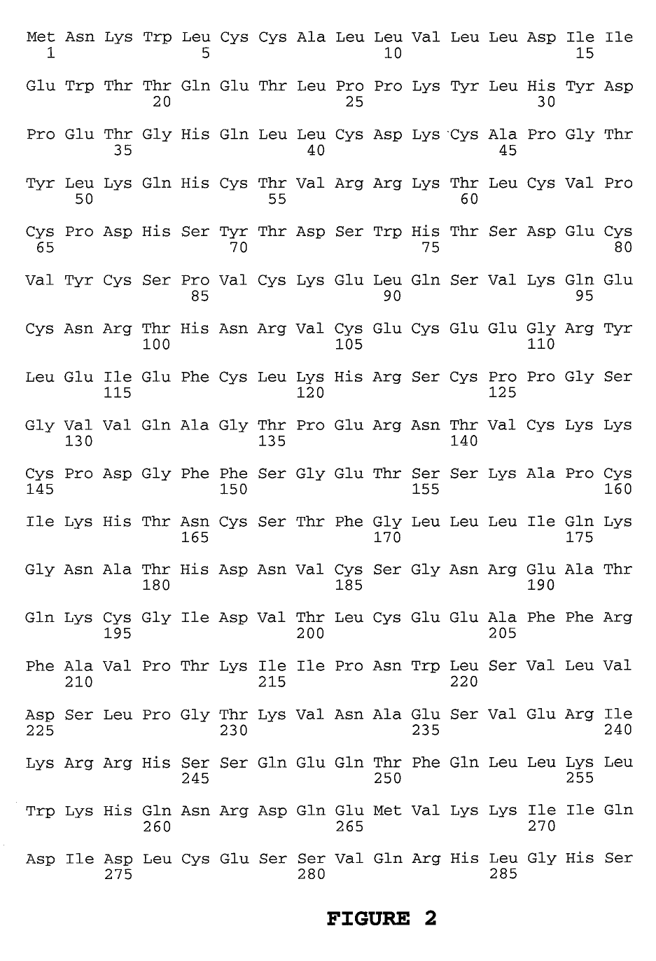

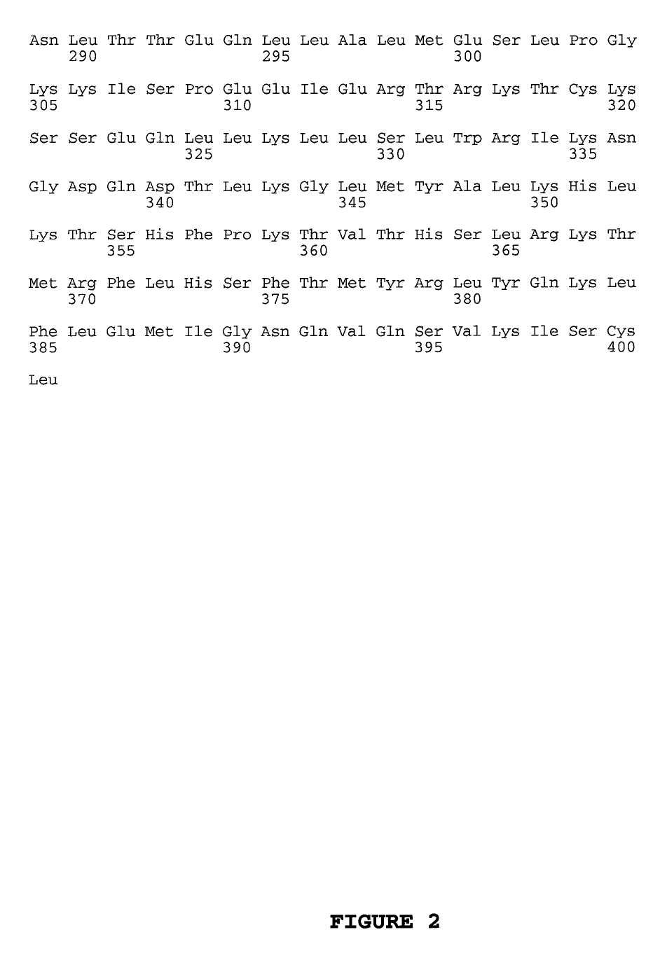

The present invention relates to OPG fusion protein compositions, methods of preparation of such compositions and uses thereof. More particularly, the present invention relates a fusion of an immunoglobulin Fc region to an OPG polypeptide. Unexpectedly, it has been observed that fusion of an Fc region to a truncated OPG polypeptide demonstrates advantages which are not seen with unfused truncated OPG polypeptides or with full-length mature OPG. (wherein full-length mature OPG has 380 amino acids, such as from residues 22 to 401 inclusive, as shown in FIG. 2 (SEQ ID NO:______)) It has been further observed that fusion of an Fc region at the carboxy terminus of an OPG polypeptide provides unexpected advantages compared to fusion of an Fc region at the amino terminus of an OPG polypeptide. Accordingly, OPG fusion proteins, and variants, fragments and derivatives thereof, as well as, related methods of use and preparation, are described in more detail below.

The term “OPG” or “OPG polypeptide” refers to a polypeptide comprising the amino acid sequence as set forth in FIG. 2 (SEQ ID NO:______) and related polypeptides described herein. Related polypeptides include allelic variants; splice variants; fragments; derivatives; substitution, deletion, and insertion variants; fusion polypeptides; and non-human homologs. OPG polypeptides may be mature polypeptides, as defined herein, and may or may not have an amino terminal methionine residue, depending on the method of preparation.

The term “OPG fusion protein” refers to an OPG protein, or OPG polypeptide which is joined to a heterologous peptide or polypeptide. The OPG fusion proteins of the invention may be prepared by any suitable means known in the art, such as by genetic or chemical fusion of OPG and heterologous peptide or polypeptide moieties. In an embodiment of the invention, the heterologous peptide or polypeptide is an Fc region of an immunoglobulin, preferably a human immunoglobulin. A heterologous peptide or protein may be joined either to the amino terminus or to the carboxy terminus of an OPG polypeptide.

The term “mature OPG polypeptide” or “mature OPG fusion polypeptide” refers to a polypeptide or a fusion polypeptide lacking a leader sequence and may also include other modifications such as proteolytic processing of the amino terminus (with or without a leader sequence) and/or the carboxy terminus, cleavage of a smaller polypeptide from a larger precursor, N-linked and/or O-linked glycosylation, and the like.

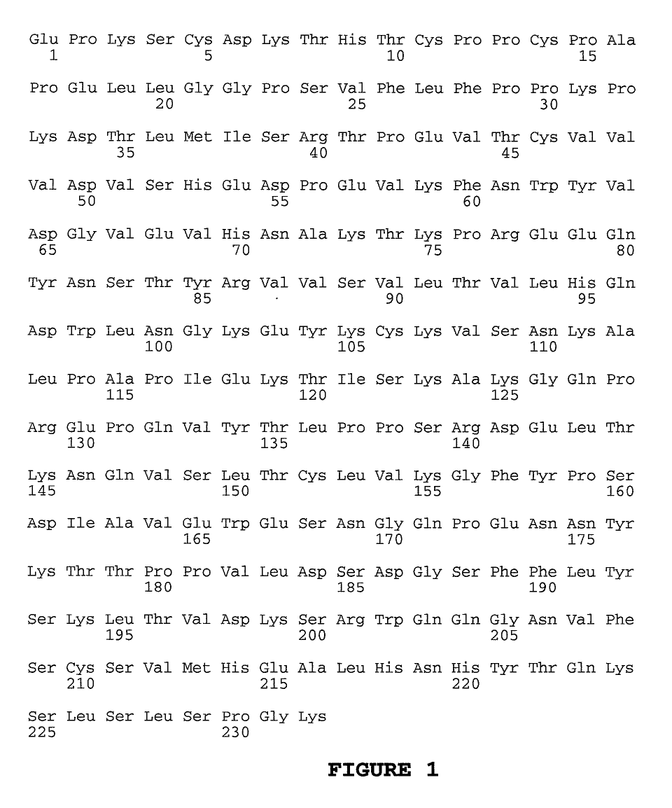

The term “Fc” refers to a molecule or sequence comprising the sequence of a non-antigen-binding portion of antibody, whether in monomeric or multimeric form. The original immunoglobulin source of an Fc is preferably of human origin and may be from any isotype, e.g., IgG, IgA, IgM, IgE or IgD. One method of preparation of an isolated Fc molecule involves digestion of an antibody with papain to separate antigen and non-antigen binding portions of the antibody. Another method of preparation of an isolated Fc molecules is production by recombinant DNA expression followed by purification of the Fc molecules so expressed. A full-length Fc consists of the following Ig heavy chain regions: C_{H}1, C_{H}2 and C_{H}3 wherein the C_{H}1 and C_{H}2 regions are typically connected by a flexible hinge region. In one embodiment, an Fc has the amino acid sequence of IgG₁ such as that shown in FIG. 1. The terms “Fc protein, “Fc sequence”, “Fc molecules, “Fc region” and “Fc portion” are taken to have the same meaning as “Fc”.

The term “fragment” when used in association with Fc or OPG polypeptides, or fusion polypeptides thereof, refers to a peptide or polypeptide that comprises less than the full length amino acid sequence of an Fc or OPG polypeptide. Such a fragment may arise, for example, from a truncation at the amino terminus, a truncation at the carboxy terminus, and/or an internal deletion of a residue(s) from the amino acid sequence. OPG or Fc fragments may result from alternative RNA splicing or from in vivo protease activity.

The term “variant” when used in association with Fc or OPG polypeptides, or with fusion polypeptides thereof, refers to a polypeptide comprising an amino acid sequence which contain one or more amino acid sequence substitutions, deletions, and/or additions as compared to native Fc or OPG polypeptide amino acid sequences. Variants may be naturally occurring or artificially constructed. Variants of the invention may be prepared from the corresponding nucleic acid molecules encoding said variants, which have a DNA sequence that varies accordingly from the DNA sequences for native Fc or OPG polypeptides.

The term “derivative” when used in association with Fc or OPG polypeptides, or with fusion polypeptides thereof, refers to Fc or OPG polypeptide variants or fragments thereof, that have been chemically modified, as for example, by covalent attachment of one or more polymers, including, but limited to, water soluble polymers, N-linked or O-linked carbohydrates, sugars, phosphates, and/or other such molecules. The derivatives are modified in a manner that is different from native Fc or OPG, either in the type or location of the molecules attached to the polypeptide. Derivatives further includes deletion of one or more chemical groups naturally attached to an Fc or OPG polypeptide.

The term “fusion” refers to joining of different peptide or protein segments by genetic or chemical methods wherein the joined ends of the peptide or protein segments may be directly adjacent to each other or may be separated by linker or spacer moieties such as amino acid residues or other linking groups.

Polypeptides

The invention provides for OPG fusion polypeptides and compositions thereof and, more particularly, provides for fusion polypeptides comprising OPG and Fc moieties. Fusions of an Fc region to an OPG polypeptide may be made at the amino terminus of OPG, that is, the carboxy terminus of an Fc region is fused to the amino terminus of OPG. These fusion proteins (and nucleic acids encoding same) are designated herein as FcOPG. It may also be desirable to fuse the carboxy terminus of OPG to the amino terminus of an Fc region. The fusion proteins (and nucleic acids encoding same) are designated herein as OPGFc.

An Fc, or a variant, fragment or derivative thereof, may be from an Ig class. In one embodiment, an Fc is from the IgG class, such as IgG₁, IgG₂, IgG₃, and IgG₄. In another embodiment, an Fc is from IgG1. An Fc may also comprise amino acid residues represented by a combination of any two or more of the Ig classes, such as residues from IgG₁ and IgG₂, or from IgG₁, IgG₂ and IgG₃, and so forth. In one embodiment, an Fc region of an OPG fusion protein has the sequence as set forth in FIG. 1 (SEQ ID NO:______) comprising hinge, C_{H}2 and C_{H}3 regions of human IgG1. (see Ellison et al., Nucleic Acids Res. 10, 4071-4079 (1982).

In addition to naturally occurring variations in Fc regions, Fc variants, fragments and derivatives may contain non-naturally occurring changes in Fc which are constructed by, for example, introducing substitutions, additions, insertions or deletions of residues or sequences in a native or naturally occurring Fc, or by modifying the Fc portion by chemical modification and the like. In general, Fc variants, fragments and derivatives are prepared such that the increased circulating half-life of Fc fusions to OPG is largely retained.

Also provided by the invention are Fc variants with conservative amino acid substitutions. The term “conservative amino acid substitution” refers to a substitution of a native amino acid residue with a normative residue such that there is little or no effect on the polarity or charge of the amino acid residue at that position. For example, a conservative substitution results from the replacement of a non-polar residue in a polypeptide with any other non-polar residue. General rules for conservative amino acid substitutions are set forth in Table I.

TABLE I

Conservative Amino Acid Substitutions

| | | Preferred |
|---|---|---|
| Original Residues | Exemplary Substitutions | Substitutions |
| Ala | Val,Leu,Ile | Val |
| Arg | Lys,Gln,Asn | Lys |
| Asn | Gln,His,Lys,Arg | Gln |
| Asp | Glu | Glu |
| Cys | Ser | Ser |
| Gln | Asn | Asn |
| Glu | Asp | Asp |
| Gly | Pro,Ala | Ala |
| His | Asn,Gln,Lys,Arg | Arg |
| Ile | Leu,Val,Met,Ala, | Leu |
| | Phe,Norleucine | |
| Leu | Norleucine,Ile, | Ile |
| | Val,Met,Ala,Phe | |
| Lys | Arg,Gln,Asn | Arg |
| Met | Leu,Phe,Ile | Leu |
| Phe | Leu,Val,Ile,Ala, | Leu |
| | Tyr | |
| Pro | Ala | Ala |
| Ser | Thr | Thr |
| Thr | Ser | Ser |
| Trp | Tyr,Phe | Tyr |
| Tyr | Trp,Phe,Thr,Ser | Phe |
| Val | Ile,Met,Leu,Phe, | Leu |
| | Ala, Norleucine | |

Conservative amino acid substitutions also encompass non-naturally occurring amino acid residues which are typically incorporated by chemical peptide synthesis rather than by synthesis in biological systems. These include peptidomimetics, and other reversed or inverted forms of amino acid moieties. Conservative modifications to the amino acid sequence (and the corresponding modifications to the encoding nucleotides) are expected to produce Fc molecules (and FcOPG fusion proteins) having functional and chemical characteristics similar to those of unmodified Fc and FcOPG proteins.

In addition to the substitutions set forth in Table I, any native residue in an Fc region (or in an FcOPG fusion protein) may also be substituted with alanine, as has been previously described for “alanine scanning mutagenesis” (Cunningham et al. Science 244, 1081-1085 (1989)).

Substantial modifications in the functional and/or chemical characteristics of an Fc molecule (and an FcOPG fusion protein) may be accomplished by selecting substitutions that differ significantly in their effect on maintaining (a) the structure of the molecular backbone in the area of the substitution, for example, as a sheet or helical conformation, (b) the charge or hydrophobicity of the molecule at the target site, or (c) the bulk of the side chain. Naturally occurring residues may be divided into groups based on common side chain properties:

1) hydrophobic: norleucine, Met, Ala, Val, Leu, Ile;

2) neutral hydrophilic: Cys, Ser, Thr;

3) acidic: Asp, Glu;

4) basic: Asn, Gln, His, Lys, Arg;

5) residues that influence chain orientation: Gly, Pro; and

6) aromatic: Trp, Tyr, Phe.

Non-conservative substitutions may involve the exchange of a member of one of these classes for a member from another class. Such substituted residues may be introduced into regions of an Fc or OPG molecule that are homologous with non-human Fc or OPG, or into the non-homologous regions of the molecule.

Cysteine residues in Fc molecules can be deleted or replaced with other amino acids to prevent formation of disulfide crosslinks. In particular, a cysteine residue at position 5 of FIG. 1 (SEQ. ID. NO. ______) may be substituted with one or more amino acids, such as alanine or serine. Alternatively, the cysteine residue at position 5 could be deleted.

An Fc fragment may be prepared by deletion of one or more amino acids at any of positions 1, 2, 3, 4 and 5 as shown in FIG. 1 (SEQ ID NO. ______). In one embodiment, the amino acid residues at positions 1-5 inclusive are deleted. Substitutions at these positions can also be made and are within the scope of this invention.

Fc variants may also be made which show reduced binding to Fc receptors which trigger effector functions such as antibody dependent cellular cytotoxicity (ADCC) and activation of complement. Such variants may include leucine at position 20 deleted or substituted with a glutamine residue, glutamate at position 103 deleted or substituted with an alanine residue, and lysines at positions 105 and 107 deleted or substituted with alanine residues (following the numbering as set forth in FIG. 1). One or more of such substitutions are contemplated.

In one embodiment, Fc variants will exhibit stronger binding to the FcRn receptor (“salvage receptor”) and a longer circulating half-life compared to native Fc. Example of such variants include amino acid substitutions at one or more of residues 33, 35-42, 59, 72, 75, 77, 95-98, 101, 172-174, 215 and 220-2:23 as shown in FIG. 1 (SEQ ID NO: ______), wherein the substitution(s) confer tighter binding of an Fc variant to the FcRn receptor.

Other Fc variants include one or more tyrosine residues replaced with, for example, phenyalanine residues. In addition, other variant amino acid insertions, deletions and/or substitutions are also contemplated and are within the scope of the present invention. Examples include Fc variants disclosed in WO96/32478 and WO97/34630 hereby incorporated by reference. Furthermore, alterations may be in the form of altered amino acids, such as peptidomimetics or D-amino acids.

An Fc protein may be also linked to an OPG protein by “linker” moieties whether chemical or amino acids of varying lengths. Such chemical linkers are well known in the art. Amino acid linker sequences can include but are not limited to:

| | |
|---|---|
| (a) | ala-ala-ala; |
| (b) | ala-ala-ala-ala; |
| (c) | ala-ala-ala-ala-ala; |
| (d) | gly-gly; |
| (e) | gly-gly-gly; |
| (f) | gly-gly-gly-gly-gly; |
| (g) | gly-gly-gly-gly-gly-gly-gly; |
| (h) | gly-pro-gly; |
| (i) | gly-gly-pro-gly-gly; |
| (j) | val; |
| (k) | ser-gly-gly-gly-gly-gly-gly-gly-gly; |
| (l) | gly-gly-ser-gly-ser-gly-ala-gly-ser-gly-ser-gly-gly-gly-ser-gly-ser-gly-gly; |
| (m) | a chemical moiety; and |
| (n) | any combination of subparts (a) |
| | through (m). |

OPG variants, fragments and derivatives are also provided by the invention and are generally as described hereinabove for Fc molecules, with the exception of the specific locations of the modified amino acid residues. OPG variants, fragments and derivatives are described in PCT WO97/23614 hereby incorporated by reference.

In a preferred embodiment, the OPG moiety of an OPG fusion protein is a carboxy-terminal truncated form of OPG. Carboxy terminal truncated forms of OPG have one or more amino acids from positions 186-401 in FIG. 2 deleted. For example, OPG truncations comprise the amino acid sequence 22-X wherein X is any residue from 185 to 400 inclusive. In another embodiment, OPG truncations comprise the amino acid sequence 22-X wherein X is any residue from 185 to 278 inclusive, or from 185 to 293 inclusive, or alternatively, from 194 to 278 inclusive, or from 194 to 293 inclusive. Fusion proteins comprising the OPG truncated polypeptides described herein encompass joining of the OPG and heterologous peptide or polypeptide moieties directly or through a spacer or linker molecule wherein the spacer or linker optionally comprises one or more amino acid residues. Variants and derivatives of the OPG truncated forms described herein are also encompassed by the invention.

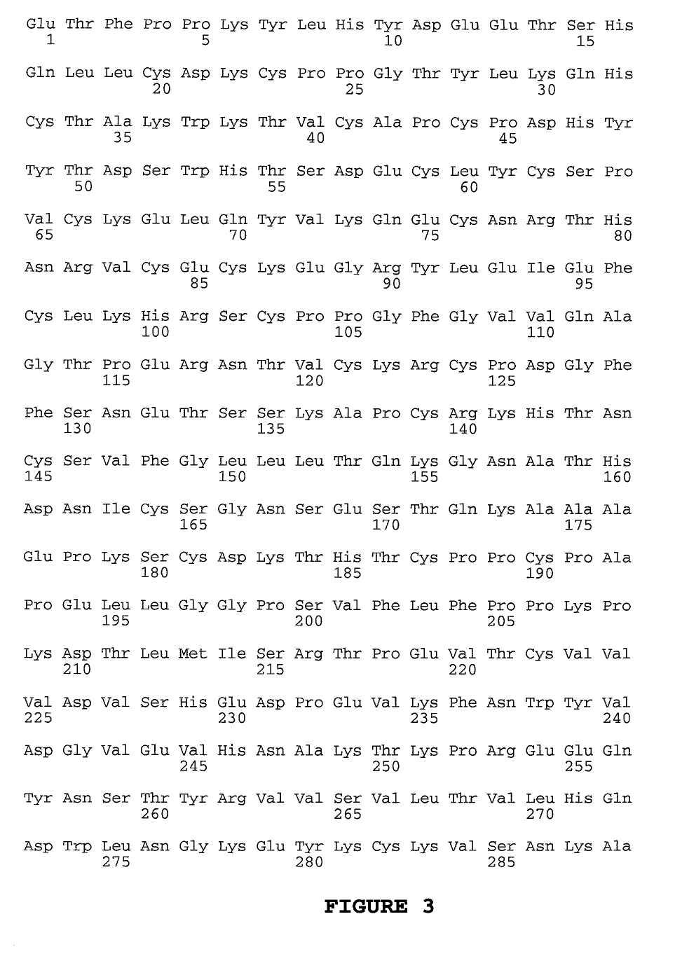

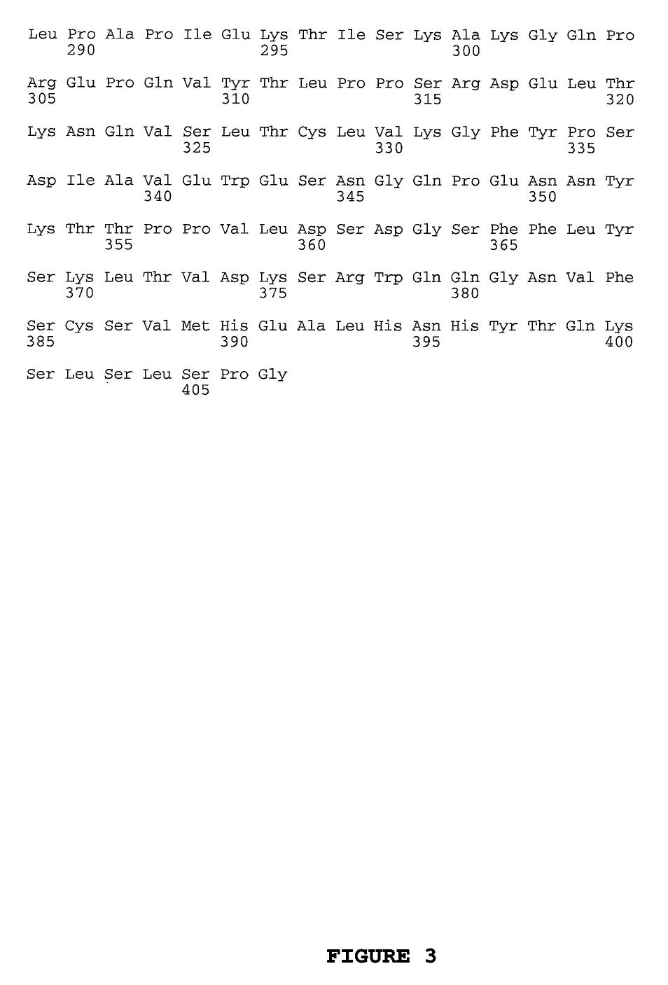

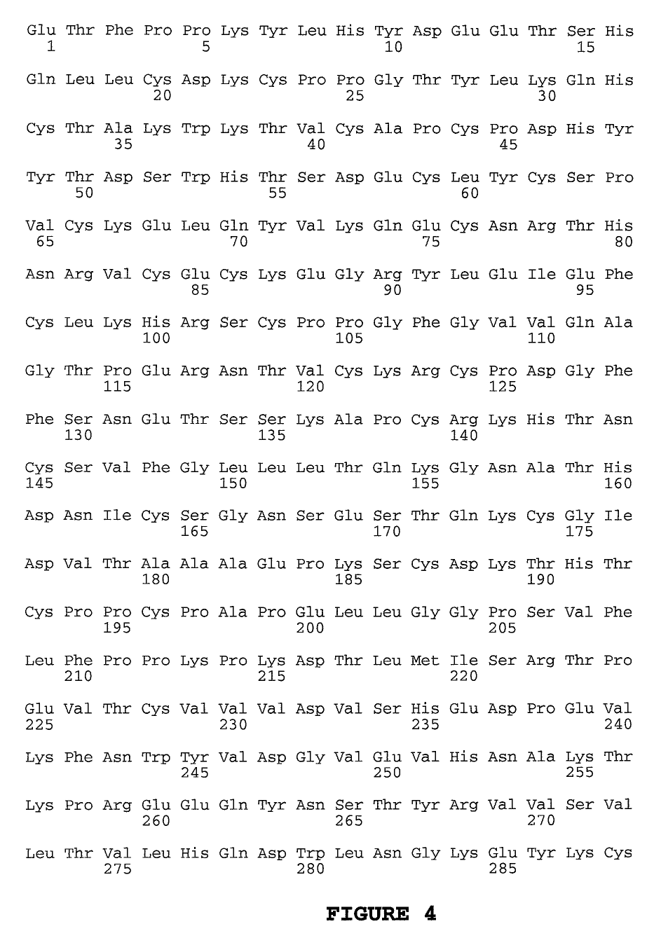

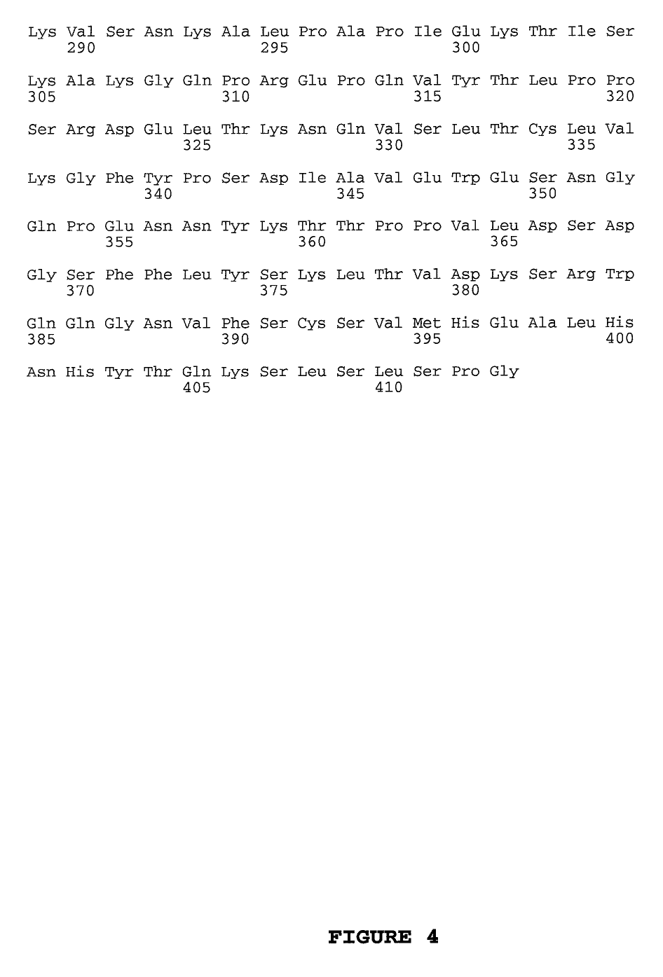

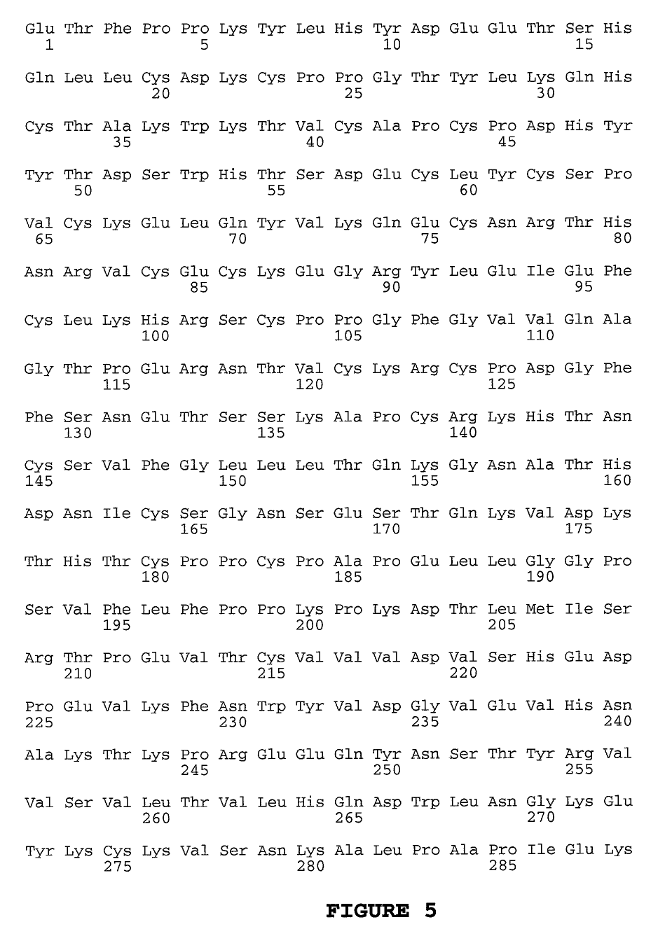

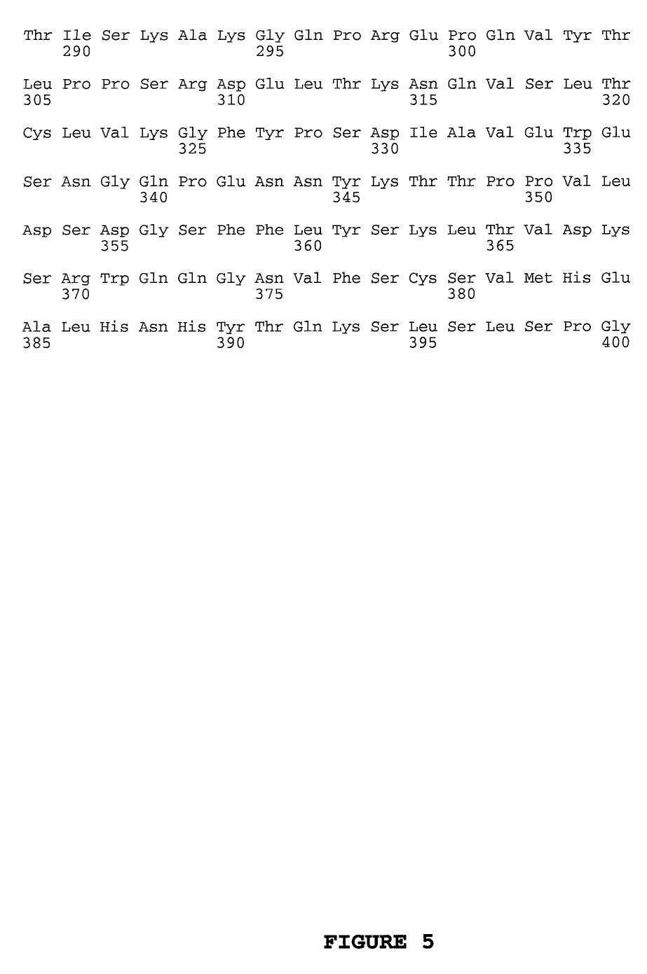

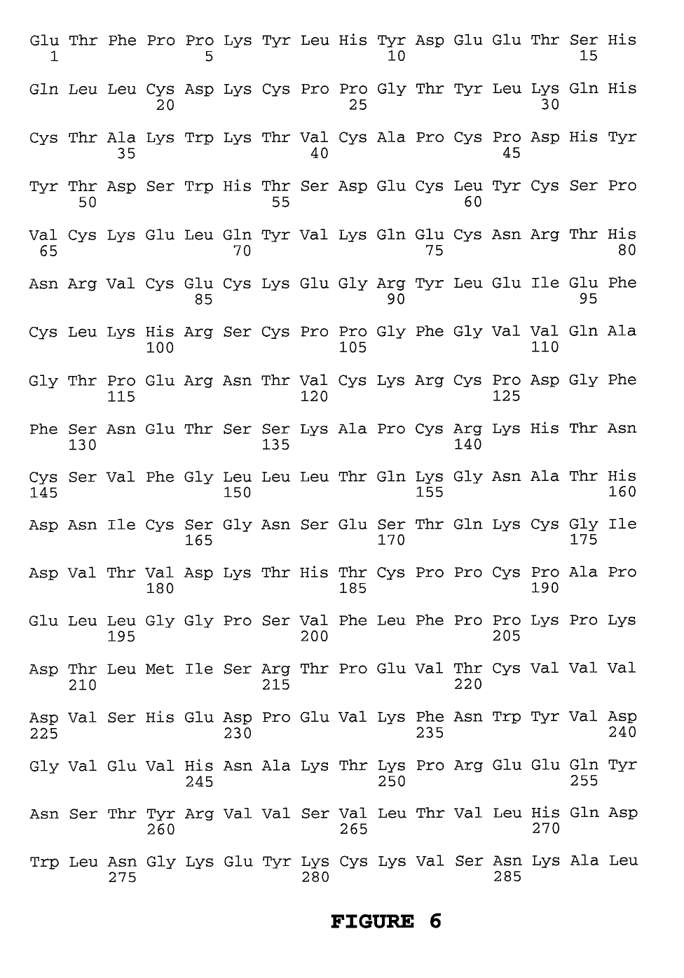

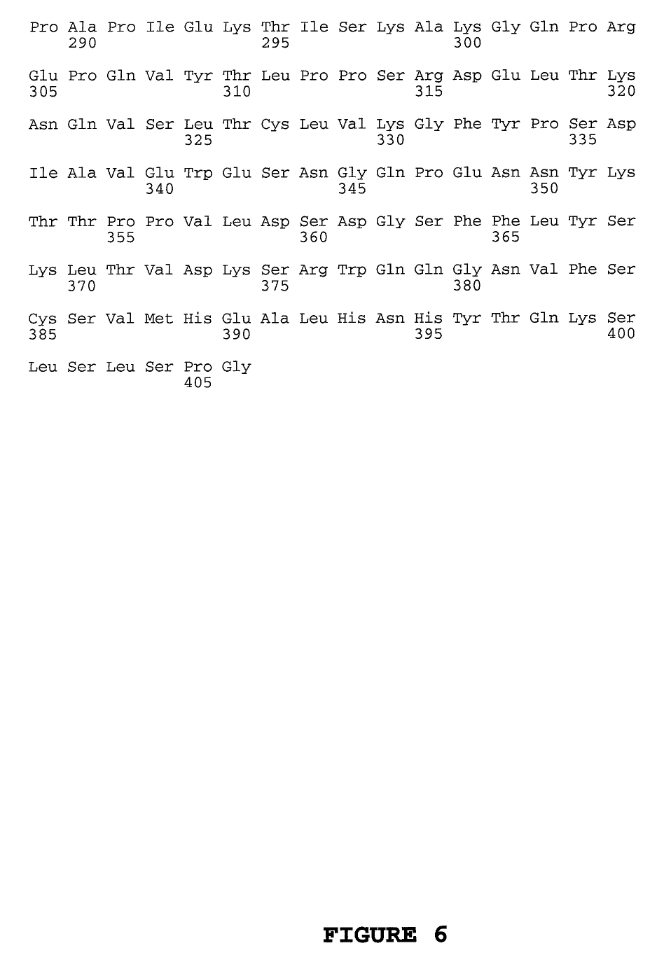

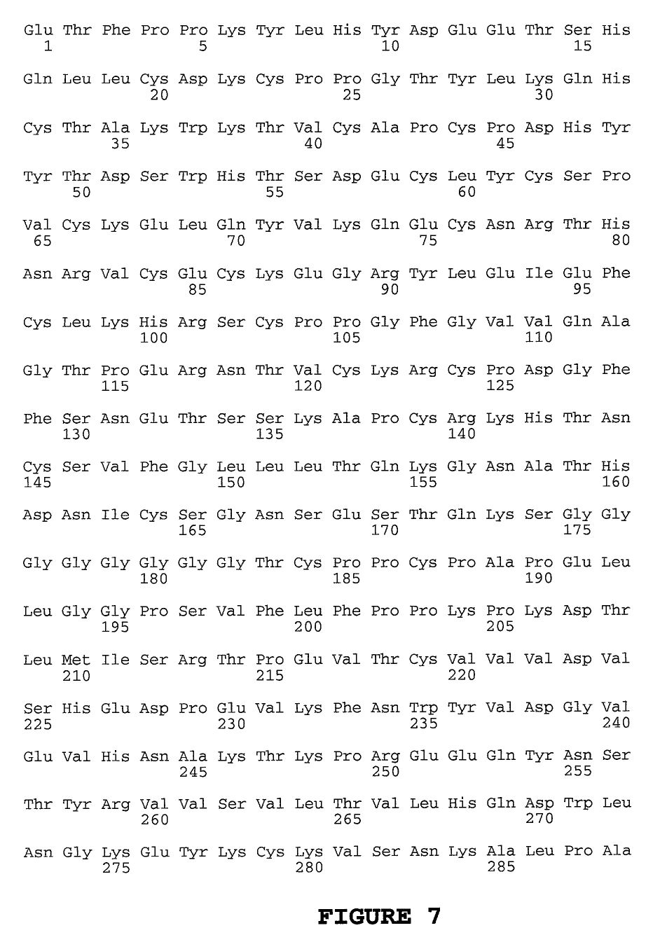

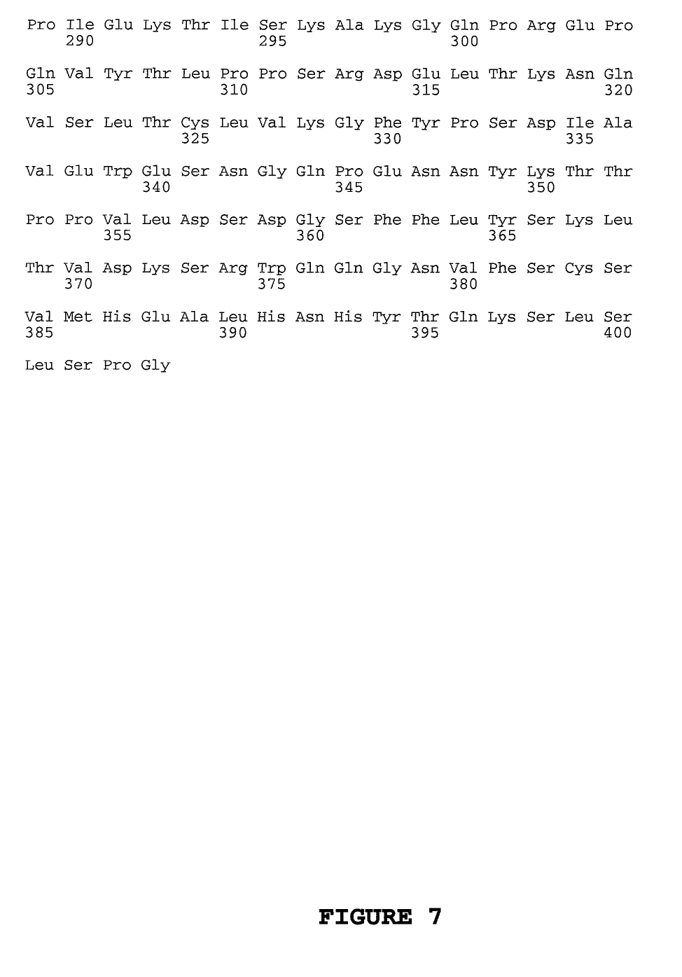

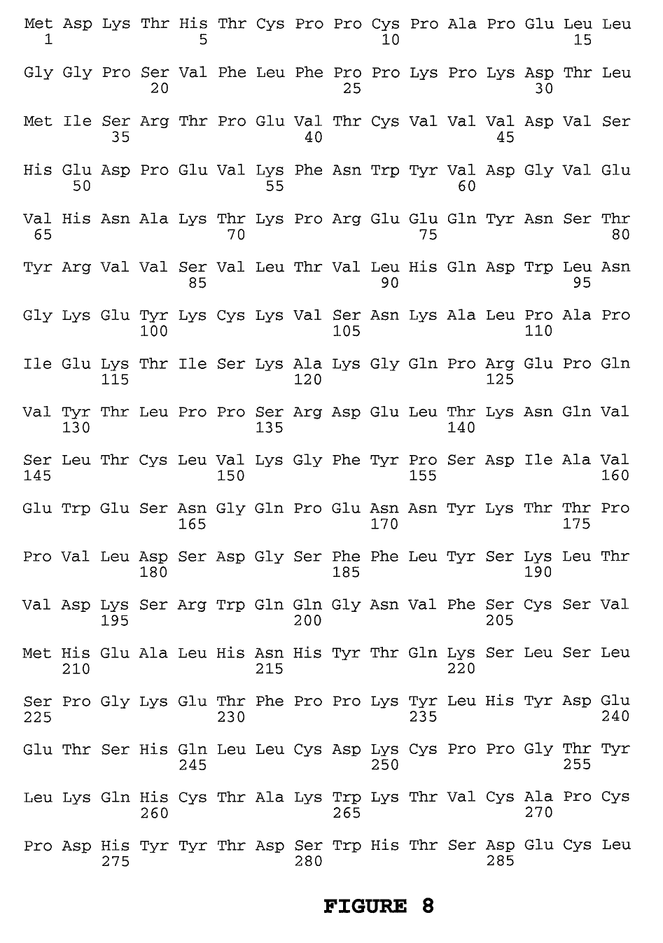

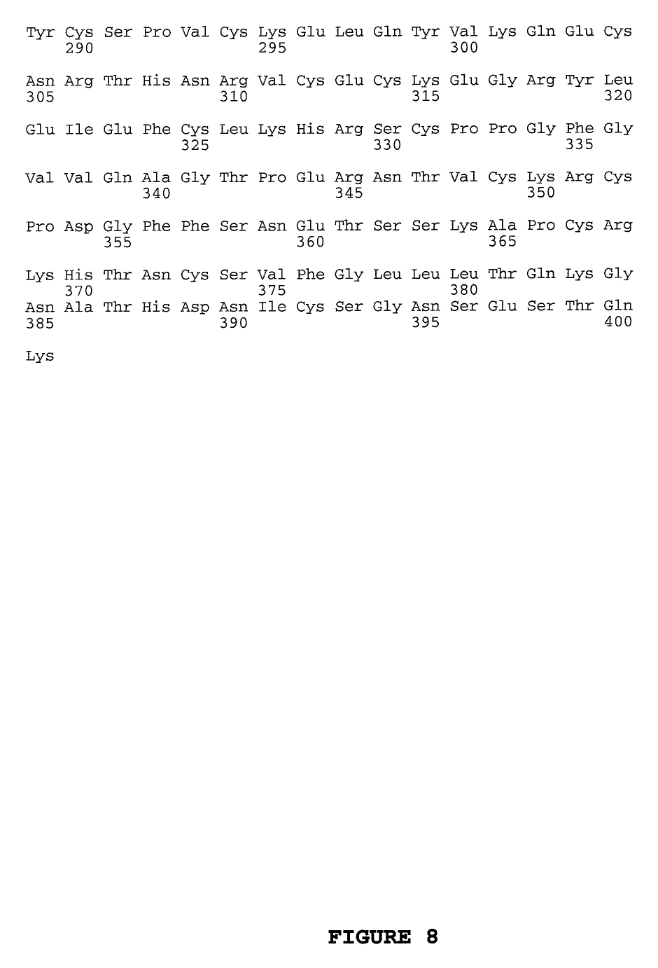

Preferred fusion proteins of the invention include those wherein the OPG moiety fused to an Fc region comprises the amino acid sequence 22-X wherein X is any residue from positions 194 to 201 inclusive using the numbering as shown in FIG. 2 (SEQ ID NO: ______). Examples of such fusion proteins include the following:

| | |
|---|---|
| OPG [22-194]-Fc | (Figure 3 and SEQ ID NO:_) |
| OPG [22-201]-Fc | (FIG. 4 and SEQ ID NO:_) |
| OPG [22-194]-FcΔC | (FIG. 5 and SEQ ID NO:_) |
| OPG [22-201]-FcΔC | (FIG. 6 and SEQ ID NO:_) |
| OPG [22-194]-FcG₁₀ | (FIG. 7 and SEQ ID NO:_) |
| metFcΔC-OPG [22-194] | (FIG. 8 and SEQ ID NO:_) |

For the preferred polypeptides listed above, the term “Fc” refers to the sequence of human IgG₁ shown in FIG. 1 (SEQ ID NO: 1), the term “FcΔC” refers to the sequence shown in FIG. 1 (SEQ ID NO: 2) Lacking amino acid residues 1-5 inclusive, and the term “FcG₁₀” refers to an Fc moiety lacking amino acid residue 1-9 inclusive and having a ser-(gly)₈ linker.

Nucleic Acid Molecules

Nucleic acid molecules encoding OPG fusion proteins, or variants, fragments or derivatives thereof, are provided for by the invention. Nucleic acid molecules of the invention may be produced using site directed mutagenesis, PCR amplification, or other appropriate methods, where the primer(s) have the desired mutations. See Sambrook et al. (Molecular Cloning: A Laboratory Manual, Cold Springs Harbor Laboratory Press, Cold Springs Harbor, N.Y. (1989)), and Ausubel et al. (Current Protocols in Molecular Biology, Wiley and Sons, N.Y. (1994)), for descriptions of mutagenesis techniques. Chemical synthesis using methods described by Engels et al. (Angew. Chem. Intl. Ed. 28, 716-734 (1989)), may also be used to prepare such variants. Other methods known to the skilled artisan may be used as well.

In certain embodiments, nucleic acid variants contain codons which have been altered for optimal expression of an OPG fusion polypeptide in a given host cell. Particular codon alterations will depend upon the OPG fusion polypeptide(s) and host cell(s) selected for expression. Such “codon optimization” can be carried out by a variety of methods, for example, by selecting codons which are preferred for use in highly expressed genes in a given host cell. Computer algorithms which incorporate codon frequency tables such as “Ecohigh. Cod” for codon preference of highly expressed bacterial genes may be used and are provided by the University of Wisconsin Package Version 9.0, Genetics Computer Group, Madison, Wis. Other useful codon frequency tables include “Celegans_high.cod”, “Celegans_low.cod”, “Drosophila_high.cod”, “Human_high.cod”, “Maize_high.cod”, and “Yeast_high.cod”. In one embodiment, codon optimatization may be carried out in either OPG or Fc moieties of the fusion polypeptide.

In another embodiment, nucleic acid molecules encode OPG fusion protein variants with conservative amino acid substitutions as defined hereinabove. For example, conservative amino acid substitutions are made in an OPG and/or in an Fc moiety of a fusion protein. Also provided for are Fc or OPG variants comprising an addition and/or a deletion of one or more N-linked or O-linked glycosylation sites, or comprising Fc or OPG polypeptide fragments as described above. It is understood that nucleic acid molecules of the invention may encode any combination of Fc and/or OPG variants, fragments, and fusion polypeptides described herein.

Vectors and Host cells

A nucleic acid molecule encoding an OPG fusion protein is inserted into an appropriate expression vector using standard ligation techniques. The vector is typically selected to be functional in the particular host cell employed (i.e., the vector is compatible with the host cell machinery such that amplification of the gene and/or expression of the gene can occur). A nucleic acid molecule encoding an Fc-OPG protein may be amplified/expressed in prokaryotic, yeast, insect (baculovirus systems) and/or eukaryotic host cells. Selection of the host cell will depend in part on whether an OPG fusion protein is to be post-translationally modified (e.g, glycosylated and/or phosphorylated). If so, yeast, insect, or mammalian host cells are preferable.

Typically, expression vectors used in any of the host cells will contain sequences for plasmid maintenance and for cloning and expression of exogenous nucleotide sequences. such sequences, collectively referred to as “flanking sequences” in certain embodiments will typically include one or more of the following nucleotides: a promoter, one or more enhancer sequences, an origin of replication, a transcriptional termination sequence, a complete intron sequence containing a donor and acceptor splice site, a leader sequence for secretion, a ribosome binding site, a polyadenylation sequence, a polylinker region for inserting the nucleic acid encoding the polypeptide to be expressed, and a selectable marker element.

Flanking sequences may be homologous (i.e., from the same species and/or strain as the host cell), heterologous (i.e, from a species other than the host cell species or strain), hybrid (i.e., a combination of flanking sequences from more than one source), synthetic, or native sequences which normally function to regulate OPG and/or Fc protein expression. As such, the source of flanking sequences may be any prokaryotic or eukaryotic organism, any vertebrate or invertebrate organism, or any plant, provided that the flanking sequences is functional in, and can be activated by, the host cell machinery.

A leader, or signal, sequence may be used to direct an OPG fusion polypeptide out of the host cell. The signal sequence is most commonly positioned directly at the 5′ end of an OPG fusion polypeptide coding region. Many signal sequences have been identified, and any of them that are functional in the selected host cell may be used in conjunction with nucleic acid sequences encoding OPG fusion proteins. For example, a signal sequence may be homologous (naturally occurring) or heterologous to an OPG or Fc gene or cDNA. Additionally, a signal sequence may be chemically synthesized using methods set forth above. In most cases, secretion of an OPG fusion polypeptide, and more particularly a fusion of OPG and Fc moieties, from the host cell via the presence of a signal peptide will result in the removal of the signal peptide from the fusion polypeptide.

The signal sequence may be a component of the vector, or it may be a part of a nucleic acid sequence encoding an OPG fusion polypeptide that is inserted into the vector. For example, native OPG DNA encodes a signal sequence at the amino terminus of the protein that is cleaved during post-translational processing of the molecule to form the mature protein. Included within the scope of this invention are OPG nucleotides with the native signal sequence as well as OPG nucleotides wherein the native signal sequence is deleted and replaced with a heterologous signal sequence. A heterologous signal sequence selected should be one that is recognized and processed, i.e., cleaved by a signal peptidase, by the host cell. The invention provides in part for a signal sequence which is the OPG signal sequence as described in WO97/23614. For prokaryotic host cells that do not recognize and process the native OPG signal sequence, the signal sequence is substituted by a prokaryotic signal sequence selected, for example, from the group of the alkaline phosphatase, penicillinase, or heat-stable enterotoxin II leaders. For yeast secretion, the native OPG signal sequence may be substituted by the yeast invertase, alpha factor, or acid phosphatase leaders. In mammalian cell expression the native signal sequence is satisfactory, although other mammalian signal sequences may be suitable.

Preferred vectors for practicing this invention are those which are compatible with bacterial, insect, and mammalian host cells. Such vectors include, inter alia, pCRII, pCR3, and pcDNA3.1 (Invitrogen Company, San Diego, Calif.), pBSII (Stratagene Company, La Jolla, Calif.), pET15b (Novagen, Madison, Wis.), pGEX (Pharmacia Biotech, Piscataway, N.J.), pEGFP-N2 (Clontech, Palo Alto, Calif.), pETL (BlueBacII; Invitrogen), pDSRα2 (PCT Publication No. WO90/14363) and pFastBacDual (Gibco/BRL, Grand Island, N.Y.).

Additional possible vectors include, but are not limited to, cosmids, plasmids or modified viruses, but the vector system must be compatible with the selected host cell. Such vectors include, but are not limited to plasmids such as Bluescript® plasmid derivatives (a high copy number ColE1-based phagemid, Stratagene Cloning Systems Inc., La Jolla Calif.), PCR cloning plasmids designed for cloning Taq-amplified PCR products (e.g., TOPO™ TA Cloning® Kit, PCR2.1® plasmid derivatives, Invitrogen, Carlsbad, Calif.), and mammalian yeast or virus vectors such as a baculovirus expression system (pBacPAK plasmid derivatives, Clontech, Palo Alto, Calif.). The recombinant molecules can be introduced into host cells via transformation, transfection, infection, electroporation, or other known techniques. After the vector has been constructed and a nucleic acid molecule encoding an OPG polypeptide has been inserted into the proper site of the vector, the completed vector may be inserted into a suitable host cell for amplification and/or polypeptide expression.

Host cells may be prokaryotic host cells (such as E. coli) or eukaryotic host cells (such as a yeast cell, an insect cell, or a vertebrate cell). The host cell, when cultured under appropriate conditions, synthesizes an OPG polypeptide which can subsequently be collected from the culture medium (if the host cell secretes it into the medium) or directly from the host cell producing it (if it is not secreted). Selection of an appropriate host cell will depend upon various factors, such as desired expression levels, polypeptide modifications that are desirable or necessary for activity, such as glycosylation or phosphorylation, and ease of folding into a biologically active molecule.

Suitable host cells or cell lines may be mammalian cells, such as Chinese hamster ovary cells (CHO) (ATCC #CCL61 and Urlaub et al., Proc. Natl. Acad. Sci. USA 77, 4216-4220 (1980)), human embryonic kidney (HEK) 293 or 293T cells (ATCC #CRL1573), or 3T3 cells (ATCC #CRL1658). The selection of suitable mammalian host cells and methods for transformation, culture, amplification, screening and product production and purification are known in the art. Other suitable mammalian cell lines, are the monkey COS-1 and COS-7 cell lines (ATCC #CRL1651), and the CV-1 cell line (ATCC #CCL70). Further exemplary mammalian host cells include primate cell lines and rodent cell lines, including transformed cell lines. Normal diploid cells, cell strains derived from in vitro culture of primary tissue, as well as primary explants, are also suitable. Candidate cells may be genotypically deficient in the selection gene, or may contain a dominantly acting selection gene. Other suitable mammalian cell lines include but are not limited to, mouse neuroblastoma N2A cells, HeLa, mouse L-929 cells, 3T3 lines derived from Swiss, Balb-c or NIH mice, BHK or HaK hamster cell lines. Each of these cell lines is known by and available to those skilled in the art.

Similarly useful as host cells suitable for the present invention are bacterial cells. For example, the various strains of E. coli (e.g., HB101, DH5a, DH10, and MC1061) are well-known as host cells in the field of biotechnology. Various strains of B. subtilis, Pseudomonas spp., other Bacillus spp., Streptomyces spp., and the like may also be employed in this method.

Many strains of yeast cells known to those skilled in the art are also available as host cells for expression of the polypeptides of the present invention. Preferred yeast cells include, for example, Saccharomyces cerivisae.

Additionally, where desired, insect cell systems may be utilized in the methods of the present invention. Such systems are described for example in Kitts et al. (Biotechniques, 14, 810-817 (1993)), Lucklow (Curr. Opin. Biotechnol., 4, 564-572 (1993)) and Lucklow et al. (J. Virol., 67, 4566-4579 (1993)). Preferred insect cells are Sf-9 and Hi5 (Invitrogen, Carlsbad, Calif.).

Transformation or transfection of an expression vector for an OPG fusion polypeptide into a selected host cell may be accomplished by well known methods including methods such as calcium chloride, electroporation, microinjection, lipofection or the DEAE-dextran method. The method selected will in part be a function of the type of host cell to be used. These methods and other suitable methods are well known to the skilled artisan, and are set forth, for example, in Sambrook et al., supra.

Polypeptide Production

Host cells comprising by transformation or transfection an OPG expression vector may be cultured using standard media well known to the skilled artisan. The media will usually contain all nutrients necessary for the growth and survival of the cells. Suitable media for culturing E. coli cells are for example, Luria Broth (LB) and/or Terrific Broth (TB). Suitable media for culturing eukaryotic cells are RPMI 1640, MEM, DMEM, all of which may be supplemented with serum and/or growth factors as required by the particular cell line being cultured. A suitable medium for insect cultures is Grace's medium supplemented with yeastolate, lactalbumin hydrolysate, and/or fetal calf serum as necessary (Gibco Life Technologies, Gaithersburg, Md.).

Typically, an antibiotic or other compound useful for selective growth of transfected or transformed cells is added as a supplement to the media. The compound to be used will be dictated by the selectable marker element present on the plasmid with which the host cell was transformed. For example, where the selectable marker element is kanamycin resistance, the compound added to the culture medium will be kanamycin; where the selectable marker element is ampicillin resistance, the compound added to the culture medium will be ampicillin.

The amount of an OPG fusion polypeptide produced by a host cell can be evaluated using standard methods known in the art. Such methods include, without limitation, Western blot analysis, SDS-polyacrylamide gel electrophoresis, non-denaturing gel electrophoresis, HPLC separation, immunoprecipitation, and/or activity assays such as DNA binding gel shift assays.

Where an OPG fusion polypeptide is prepared without a tag attached, and no antibodies are available, other well known procedures for purification can be used. Such procedures include, without limitation, ion exchange chromatography, molecular sieve chromatography, HPLC, native gel electrophoresis in combination with gel elution, and preparative isoelectric focusing (“Isoprime” machine/technique, Hoefer Scientific). In some cases, two or more of these techniques may be combined to achieve increased purity.

If an OPG fusion polypeptide is produced intracellularly, the intracellular material (including inclusion bodies for gram-negative bacteria) can be extracted from the host cell using any standard technique known to the skilled artisan. For example, the host cells can be lysed to release the contents of the periplasm/cytoplasm by French press, homogenization, and/or sonication followed by centrifugation.

If an OPG fusion polypeptide has formed inclusion bodies in the cytosol, the inclusion bodies can often bind to the inner and/or outer cellular membranes and thus will be found primarily in the pellet material after centrifugation. The pellet material can then be treated at pH extremes or with chaotropic agent such as a detergent, guanidine, guanidine derivatives, urea, or urea derivatives in the presence of a reducing agent such as dithiothreitol at alkaline pH or tris carboxyethyl phosphine at acid pH to release, break apart, and solubilize the inclusion bodies. An OPG polypeptide in its now soluble form can then be analyzed using gel electrophoresis, immunoprecipitation or the like. If it is desired to isolate an OPG fusion polypeptide, isolation may be accomplished using standard methods such as those set forth below and in Marston et al. (Meth. Enz., 182, 264-275 (1990)).

In some cases, an OPG fusion polypeptide may not be biologically active upon isolation. Various methods for “refolding” or converting the polypeptide to its tertiary structure and generating disulfide linkages, can be used to restore biological activity. Such methods include exposing the solubilized polypeptide to a pH usually above 7 and in the presence of a particular concentration of a chaotrope. The selection of chaotrope is very similar to the choices used for inclusion body solubilization, but usually the chaotrope is used at a lower concentration and is not necessarily the same as chaotropes used for the solubilization. In most cases the refolding/oxidation solution will also contain a reducing agent or the reducing agent plus its oxidized form in a specific ratio to generate a particular redox potential allowing for disulfide shuffling to occur in the formation of the protein's cysteine bridge(s). Some of the commonly used redox couples include cysteine/cystamine, glutathione (GSH)/dithiobis GSH, cupric chloride, dithiothreitol(DTT)/dithiane DTT, and 2-mercaptoethanol(bME)/dithio-b(ME). In many instances, a cosolvent may be used or may be needed to increase the efficiency of the refolding and the more common reagents used for this purpose include glycerol, polyethylene glycol of various molecular weights, arginine and the like.

Derivatives

The present OPG fusion proteins, and variants and fragments thereof, are derivatized by attachment of one or more chemical moieties. As an example, a fusion of OPG and Fc polypeptides may be derivatized on either OPG or Fc moieties, or both. These chemically modified derivatives may be further formulated for intraarterial, intraperitoneal, intramuscular subcutaneous, intravenous, oral, nasal, pulmonary, topical or other routes of administration as discussed below. Chemical modification of biologically active proteins has been found to provide additional advantages under certain circumstances, such as increasing the stability and circulation time of the therapeutic protein and decreasing immunogenicity. See, U.S. Pat. No. 4,179,337. For a review, see Abuchowski et al., in Enzymes as Drugs. (J. S. Holcerberg and J. Roberts, eds. pp. 367-383 (1981)); Francis et al., supra.

The chemical moieties suitable for such derivatization may be selected from among various water soluble polymers. One skilled in the art will be able to select the desired polymer based on such considerations as whether the polymer/protein conjugate will be used therapeutically, and if so, the desired dosage, circulation time, resistance to proteolysis, and other considerations. For the present proteins, the effectiveness of the derivatization may be ascertained by administering the derivative, in the desired form (i.e., by osmotic pump, or, more preferably, by injection or infusion, or, further formulated for oral, pulmonary or nasal delivery, for example), and observing biological effects as described herein.

The water soluble polymer may be selected from the group consisting of, for example, polyethylene glycol, copolymers of ethylene glycol/propylene glycol, carboxymethylcellulose, dextran, polyvinyl alcohol, polyvinyl pyrolidone, poly-1, 3-dioxolane, poly-1,3,6-trioxane, ethylene/maleic anhydride copolymer, polyaminoacids (either homopolymers or random copolymers), and dextran or poly(n-vinyl pyrolidone)polyethylene glycol, propylene glycol homopolymers, polypropylene oxide/ethylene oxide co-polymers, polyoxyethylated polyols and polyvinyl alcohol. Polyethylene glycol propionaldenhyde may have advantages in manufacturing due to its stability in water. Also, succinate and styrene may also be used. In addition, polyaminoacids may be selected from the group consisting of serum album (such as human serum albumin), or other polyaminoacids, e.g. lysines.

The polymer may be of any molecular weight, and may be branched or unbranched. For polyethylene glycol, the preferred molecular weight is between about 2 kDa and about 100 kDa (the term “about” indicating that in preparations of polyethylene glycol, some molecules will weigh more, some less, than the stated molecular weight) for ease in handling and manufacturing.

The number of polymer molecules attached to an OPG fusion polypeptide may vary, and one skilled in the art will be able to ascertain the effect on function. One may mono-derivatize, or may provide for a di-, tri-, tetra- or some combination of derivatization, with the same or different chemical moieties (e.g., polymers, such as different weights of polyethylene glycols). The proportion of polymer molecules to protein (or peptide) molecules will vary, as will their concentrations in the reaction mixture. In general, the optimum ratio (in terms of efficiency of reaction in that there is no excess unreacted protein or polymer) will be determined by factors such as the desired degree of derivatization (e.g., mono, di-, tri-, etc.), the molecular weight of the polymer selected, whether the polymer is branched or unbranched, and the reaction conditions.

The chemical moieties should be attached to an OPG fusion protein with consideration of effects on functional or antigenic domains of the protein. There are a number of attachment methods available to those skilled in the art. (EP 0401384 herein incorporated by reference (coupling PEG to G-CSF); Malik et al., Exp. Hematol. 20, 1028-1035 (1992) (reporting pegylation of GM-CSF using tresyl chloride)). For example, polyethylene glycol may be covalently bound through amino acid residues having a free amino group (e.g., lysine, arginine or N-terminal residue) or a free carboxyl group (e.g., glutamic acid, aspartic acid, or C-terminal residue). Amino acid residues having a free sulfhydryl group (e.g., cysteine) may also be used. Preferred for therapeutic purposes is attachment at an amino group, such as attachment at the N-terminus or lysine group. Attachment at residues important for receptor binding should be avoided if receptor binding is desired.

One may specifically desire N-terminally chemically modified OPG fusion protein. Using polyethylene glycol as an example of the chemical moiety, a preparation of substantially N-terminally pegylated OPG fusion polypeptide may be obtained by derivatizing the polypeptide at free amino groups and separating N-terminally pegylated material from a population of pegylated protein molecules. Alternatively, selective N-terminal chemical modification may be accomplished by reductive alkylation which exploits differential reactivity of different types of primary amino groups (lysine versus the N-terminal) available for derivatization in a particular protein. Under the appropriate reaction conditions, substantially selective derivatization of the protein at the N-terminus with a carbonyl group containing polymer is achieved. Polyethylene glycol propionaldehyde, containing a single reactive aldehyde, may be used.

An N-terminally monopegylated derivative is preferred for ease in production of a therapeutic. N-terminal pegylation ensures a homogenous product as characterization of the product is simplified relative to di-, tri- or other multi-pegylated products. The use of reductive alkylation for preparation of an N-terminal product is preferred for ease in commercial manufacturing.

Uses of the Polypeptide

The fusion polypeptides of the invention are used in the prevention and/or treatment of loss of bone mass. Bone loss is manifested in a variety of condition including the following:

Osteoporosis, such as primary osteoporosis, endocrine osteoporosis (hyperthyroidism, hyperparathryoidism, Cushing's syndrome, and acromegaly), hereditary and congenital forms of osteoporosis (osteogenesis imperfecta, homocystinuria, Menkes' syndrome, and Riley-Day syndrome) and osteoporosis due to immobilization of extremities; Paget's disease of bone (osteitis deformans) in adults and juveniles; osteomyelitis, or an infectious lesion in bone, leading to bone loss; hypercalcemia resulting from solid tumors (breast, lung and kidney) and hematologic malignacies (multiple myeloma, lymphoma and leukemia), idiopathic hypercalcemia, and hypercalcemia associated with hyperthryoidism, hyperparathyroidism, sarcoid, and renal function disorders; osteopenia following surgery, induced by steroid administration, and associated with disorders of the small and large intestine and with chronic hepatic and renal diseases; osteonecrosis, or bone cell death, associated with traumatic injury or nontraumatic necrosis associated with Gaucher's disease, sickle cell anemia, systemic lupus erythematosus and other conditions; bone loss due to rheumatoid arthritis; periodontal bone loss; osteolytic metastasis; osteolytic arthritis; and prosthetic loosening.

In an embodiment of the invention, an OPG fusion polypeptide, by virtue of increased activity and circulating half-life, is advantageously used to treat bone loss, and especially bone loss resulting from osteolytic destruction of bone caused by malignant or metastatic tumors. OPG fusion polypeptides of the invention may be used to treat bone loss associated with breast, prostate, thyroid, kidney, lung, esophogeal, rectal, bladder, cervical, ovarian and liver cancers as well as cancer of the gastrointestional tract. Also included is bone loss associated with certain hematological malignancies such as multiple myeloma and lymphomas such as Hodgkin's Disease.

Pharmaceutical Compositions

The present invention also provides for pharmaceutical compositions of OPG fusion proteins, and variants, fragments and derivatives thereof. Such pharmaceutical compositions may be for administration for injection, or for oral, pulmonary, nasal, transdermal or other forms of administration. In general, comprehended by the invention are pharmaceutical compositions comprising effective amounts of an OPG fusion protein of the invention together with pharmaceutically acceptable diluents, preservatives, solubilizers, emulsifiers, adjuvants and/or carriers. An effective or a therapeutically effective amount of an OPG fusion protein is an amount sufficient to reduce the amount or rate of bone loss as determined by assays and procedures described below.

Pharmaceutical compositions of the invention include diluents of various buffer content (e.g., Tris-HCl, acetate, phosphate), pH and ionic strength; additives such as detergents and solubilizing agents (e.g., Tween 80, Polysorbate 80), anti-oxidants (e.g., ascorbic acid, sodium metabisulfite), preservatives (e.g., Thimersol, benzyl alcohol) and bulking substances (e.g., lactose, mannitol); incorporation of the material into particulate preparations of polymeric compounds such as polylactic acid, polyglycolic acid, etc. or into liposomes. Hylauronic acid may also be used, and this may have the effect of promoting sustained duration in the circulation. Such compositions may influence the physical state, stability, rate of in vivo release, and rate of in vivo clearance of the present proteins and derivatives. See, e.g., Remington's Pharmaceutical Sciences, 18th Ed., Mack Publishing Co., Easton, Pa. (1990), pp. 1435-1712, which are herein incorporated by reference. The compositions may be prepared in liquid form, or may be in dried powder, such as lyophilized form. Implantable sustained release formulations are also contemplated, as are transdermal formulations.

Contemplated for use herein are oral solid dosage forms, which are described generally in Remington's Pharmaceutical Sciences, 18th Ed. 1990 (Mack Publishing Co. Easton, Pa. 18042) at Chapter 89, which is herein incorporated by reference. Solid dosage forms include tablets, capsules, pills, troches or lozenges, cachets or pellets. Also, liposomal or proteinoid encapsulation may be used to formulate the present compositions (as, for example, proteinoid microspheres reported in U.S. Pat. No. 4,925,673). Liposomal encapsulation may be used and the liposomes may be derivatized with various polymers (e.g., U.S. Pat. No. 5,013,556). A description of possible solid dosage forms is given by Marshall, K. In: Modern Pharmaceutics Edited by G. S. Banker and C. T. Rhodes Chapter 10, 1979, herein incorporated by reference. In general, the formulation will include the OPG fusion protein, or a variant, fragment or derivative thereof, and inert ingredients which allow for protection against the stomach environment, and release of the biologically active material in the intestine.

An OPG fusion protein may optionally be chemically modified so that oral delivery of the derivative is efficacious. Generally, the chemical modification contemplated is the attachment of at least one moiety to the protein (or peptide) molecule itself, where said moiety permits (a) inhibition of proteolysis; and (b) uptake into the blood stream from the stomach or intestine. Also desired is the increase in overall stability of the protein and increase in circulation time in the body. Examples of such moieties include polyethylene glycol, copolymers of ethylene glycol and propylene glycol, carboxymethyl cellulose, dextran, polyvinyl alcohol, polyvinyl pyrrolidone and polyproline. Abuchowski and Davis, Soluble Polymer-Enzyme Adducts. In: “Enzymes as Drugs”, Hocenberg and Roberts, eds., Wiley-Interscience, New York, N.Y., (1981), pp 367-383; Newmark, et al., J. Appl. Biochem. 4: 185-189 (1982). Other polymers that could be used are poly-1,3-dioxolane and poly-1,3,6-tioxocane. Preferred for pharmaceutical usage, as indicated above, are polyethylene glycol moieties.

To ensure resistance to degradation in the stomach following oral administration, a coating impermeable to at least pH 5.0 is essential. Examples of the more common inert ingredients that are used as enteric coatings for oral formulations are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

An OPG fusion protein may be included in a formulation as fine multiparticulates in the form of granules or pellets of particle size about 1 mm. The formulation of the material for capsule administration could also be as a powder, lightly compressed plugs or even as tablets.

Pharmaceutical compositions of the invention include diluents such as carbohydrates, especially mannitol, α-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in solid dosage formulations. Materials used as disintegrates include but are not limited to starch including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used for hard tablets and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

Lubricants that may be added to the formulation include, but are not limited to, stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of an OPG fusion protein composition, a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. Potential nonionic detergents that could be used as surfactants include lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the protein or derivative either alone or as a mixture in different ratios.

Additives which potentially enhance uptake of a polypeptide are for instance the fatty acids oleic acid, linoleic acid and linolenic acid.

A controlled release formulation may be desirable. An OPG fusion protein may be incorporated into an inert matrix which permits release by either diffusion or leaching mechanisms e.g., gums. Slowly degenerating matrices may also be incorporated into the formulation, e.g., alginates, polysaccahrides. Another form of a controlled release of this therapeutic is by a method based on the Oros therapeutic system (Alza Corp.), i.e., the drug is enclosed in a semipermeable membrane which allows water to enter and push drug out through a single small opening due to osmotic effects. Some enteric coatings also have a delayed release effect.

Other coatings may be used for the formulation. For example, a film coated tablet may comprise materials from two different groups. The first group includes nonenteric materials such as methyl cellulose, ethyl cellulose, hydroxyethyl cellulose, methylhydroxy-ethyl cellulose, hydroxypropyl cellulose, hydroxypropyl-methyl cellulose, sodium carboxy-methyl cellulose, providone and the polyethylene glycols. The second group consists of the enteric materials that are commonly esters of phthalic acid. A mix of materials might be used to provide the optimum film coating. Film coating may be carried out in a pan coater or in a fluidized bed or by compression coating.

Also contemplated herein is pulmonary delivery of the present protein (or derivatives thereof). The protein (or derivative) is delivered to the lungs of a mammal while inhaling and traverses across the lung epithelial lining to the blood stream. (Other reports of this include Adjei et al., Pharmaceutical Research 7: 565-569 (1990); Adjei et al., International Journal of Pharmaceutics 63: 135-144 (1990) (leuprolide acetate); Braquet et al., Journal of Cardiovascular Pharmacology 13 (suppl. 5): s.143-146 (1989)(endothelin-1); Hubbard et al., Annals of Internal Medicine 3: 206-212 (1989)(α1-antitrypsin); Smith et al., J. Clin. Invest. 84: 1145-1146 (1989) (α1-proteinase); Oswein et al., “Aerosolization of Proteins”, Proceedings of Symposium on Respiratory Drug Delivery II, Keystone, Colo., March, 1990 (recombinant human growth hormone); Debs et al., The Journal of Immunology 140: 3482-3488 (1988)(interferon γ and tumor necrosis factor α) and U.S. Pat. No. 5,284,656 (granulocyte colony stimulating factor).

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp., Bedford, Mass.

All such devices require the use of formulations suitable for the dispensing of a polypeptide or a polypeptide product. Typically, each formulation is specific to the type of device employed and may involve the use of an appropriate propellant material, in addition to diluents, adjuvants and/or carriers useful in therapy.

An OPG fusion protein (or derivative) should most advantageously be prepared in particulate form with an average particle size of less than 10 μm, most preferably 0.5 to 5 μm, for most effective delivery to the distal lung.

Carriers include carbohydrates such as trehalose, mannitol, xylitol, sucrose, lactose, and sorbitol. Other ingredients for use in formulations may include DPPC, DOPE, DSPC and DOPC. Natural or synthetic surfactants may be used. Polyethylene glycol may be used (even apart from its use in derivatizing an OPG fusion protein). Dextrans, such as cyclodextran, may be used. Bile salts and other related enhancers may be used. Cellulose and cellulose derivatives may be used. Amino acids may be used, such as use in a buffer formulation. The use of liposomes, microcapsules or microspheres, inclusion complexes, or other types of carriers is contemplated.

Nasal delivery of an OPG fusion protein is also contemplated. Nasal delivery allows the passage of the protein to the blood stream directly after administering the therapeutic product to the nose, without the necessity for deposition of the product in the lung. Formulations for nasal delivery include those with dextran or cyclodextran. Delivery via transport across other mucus membranes is also contemplated.

Dosages

OPG fusion polypeptides of the invention are administered in a therapeutically effective amount to prevent and/or treat loss of bone associated with metastatic bone disease. A “therapeutically effective amount” of an OPG fusion polypeptide is that amount which reduces the rate and/or extent of loss of bone mass. Bone mass is measured by a variety of known methods such as single photon absorptiometry (SPA), dual photon absorptiomerty (DPA), dual energy X-ray absorptiometry (DEXA), quantitative computed tomography (QCT), and ultrasonography (See Johnston et al. in Primer on the Metabolic Bone Disease and Disorders of Mineral Metabolism, 2^{nd} ed., M. J. Favus, ed. Raven Press pp. 137-146). One skilled in the art can use these methods to determine a therapeutically effective amount of an OPG fusion polypeptide. A therapeutically effective amount may also be determined by measuring changes in biochemical markers for bone turnover, such as serum osteocalcin, serum alkaline phosphatase, serum procollagen I extension peptides, urinary or serum C-terminal or N-terminal telopeptide of collagen, urinary calcium, hydroxyproline and urinary pyridinoline and deoxypyridinoline. It is generally recognized that a decrease in the levels of the aforementioned biochemical markers indicates that bone resorption is decreased and bone loss is being reduced. Alternatively, a therapeutically effective amount of an OPG fusion polypeptide may also be determined by measuring a change in the mechanical strength of bone, in particular an increase in torsional (twisting) strength of bone.

In general, a therapeutically effective amount of an OPG fusion polypeptide is from about 0.1 mg/kg to about 10 mg/kg, preferably from about 1 mg/kg to about 10 mg/kg. By virtue of the increased half-life of an OPG fusion polypeptide, especially a fusion of OPG to an immunoglobulin Fc region, the frequency of administration will be less than with unmodified OPG, such as a mature full-length OPG polypeptide. An OPG fusion polypeptide will be administered about one time per month, or alternatively one time every two months, or one time every three months. It will be appreciated that the exact dosage and frequency of administration will depend upon several factors, including formulation, route of administration, condition being treated, and so forth, and may be readily determined by the skilled worker.

The amount of OPG fusion protein which has been administered may be determined using diagnostic assays for the fusion protein. Such diagnostic assays may be in the form of an antibody assay, such as an antibody sandwich assay, wherein the antibody specifically binds to an OPG fusion protein but does not bind to endogenous, naturally circulating OPG. Antibody based assays for determining OPG fusion protein levels may be carried out in a variety of formats that are known to one skilled in the art.

The following examples are offered to more fully illustrate the invention, but are not construed as limiting the scope thereof.

### EXAMPLE 1

### Construction and Expression of OPG Polypeptides and OPG Fusion Polypeptides

Construction of a recombinant plasmid encoding OPG[1-401] as shown in FIG. 2 (SEQ ID NO:______) is described in WO97/23614 hereby incorporated by reference. This plasmid was used in mammalian host cells to produce a mature full-length OPG polypeptide having amino acid residues 22 to 401 inclusive as shown in FIG. 2 (SEQ ID NO:______). Plasmids encoding OPG[1-201] and OPG[1-201]-Fc polypeptides were constructed generally as described in WO97/23614. These plasmids were used to produce OPG[22-201] and OPG[22-201]-Fc polypeptides.

OPG[1-194] was constructed by PCR using oligonucleotides 1745-92 and 1789-04 and OPG cDNA as a template. The sense primer (1745-92) created an XbaI site for cloning and a consensus Kozak sequence before the initiator ATG. The antisense primer (1789-04) placed a stop codon after amino acid residue 194 and a SalI restriction site for cloning. This PCR product was cloned into pDSRα19 to generate pDSRα19-huOPG[1-194] for mammalian expression of an OPG[22-194] polypeptide.

OPG [1-293] was constructed by PCR using oligonucleotides 1745-92 and 1745-94 and OPG cDNA as a template. The sense primer (1745-92) created an XbaI site for cloning and a consensus Kozak sequence before the initiator ATG. The antisense primer (1745-94) placed a stop codon after amino acid residue 293 and a SalI restriction site for cloning. This PCR product was cloned into pDSRα19 to generate pDSRα19:huOPG[1-293] for mammalian expression of OPG[22-293].

```
1745-92
5′-AAG TCTAGA CCACC ATG AAC AAG TTG CTG T-3′
                XbaI Kozak OPG coding
1745-94
5′-GCTA GTCGA CTA CTC GAA GGT GAG GTT AGC AT-3′
                  SalI * OPG coding
1789-04
5′-ATCT GTCGA CTA TTT TTG AGT TGA TTC AC-3′
                  SalI * OPG coding
```

Construction of OPG[1-194]-FcΔc

The plasmid pDSRα19:OPG[1-194]-FcΔC was constructed from the plasmid pDSRα2:OPG[1-201]-Fc using PCR methods to remove an unpaired cysteine at the 3′ end of the OPG segment and an unpaired cysteine at the 5′ end of the Fc segment. This clone was then used as a template for PCR to obtain the OPG domain. The 5′ OPG primer incorporated an XbaI site (TCTAGA) for cloning and a “CCACC” Kozak sequence before the initiator Met codon. The 3′ OPG primer incorporated a SalI site (GTCGAC) for cloning the Fc domain. The PCR generated a 592 bp fragment of the OPG gene, encoding the first 194 amino acid residues of the OPG protein. The PCR product was cut with XbaI and SalI and cloned into pDSRα19 to generate the final construct, called plasmid p615.

```
Sense OPG primer (1745-92):
5′-AAG TCTAGA CCACC ATG AAC AAG TTG CTG T-3′
        XbaI Site Kozak OPG coding
Antisense OPG primer (1775-27):
5′-CACGC GTCGAC TTT TTG ACT TGA TTC ACT GTT TCC-3′
              SalI Site OPG coding
```

The clone pDSRα2/OPG[1-201]-Fc was used as a template to obtain the Fc domain. The PCR generated the Fc carboxy-terminal 227 aa including the hinge, C_{H}2 and C_{H}3 domains. The 5′Fc primer incorporated a SalI site (encoding “VD”) and the 3′ Fc primer incorporated a XhoI site (CTCGAG) after the Fc termination codon. The Fc PCR product was cloned into the SalI site of p615 to yield pDSRα19:OPG[1-194]-FcΔC which produces OPG[22-194]-FcdC upon expression in mammalian cells. The fusion protein contains an extra valine at the Fc-OPG junction. The XhoI site is lost in the ligation.

```
Sense Fc primer (1476-25)
5′-AATCT GTCGAC AAA ACT CAC ACA TGC-3′
                SalI Site Fc Coding
Antisense Fc primer (1504-63)
5′-CCATG CTCGAG TTA TCA TTT ACC CGG AGA CAG G-3′
              XhoI Site * Fc coding
```

Construction of OPG[1-194]-FcG10

An Fc region with a G10 hinge (one serine and eight glycine residues) was constructed by PCR using primers 1775-30 and 1504-63 and OPG[1-201]-Fc cDNA as a template. The product was subcloned into pCRscript (pCRscriptFcG10BspE) and sequenced. OPG[1-194] was obtained by PCR using primers 1745-92 and 1790-72 and OPG[1-201]-Fc cDNA as a template. The PCR product was subcloned into pCRScript and sequenced. An Xba/BspEI fragment containing OPG[1-194] sequence and a BspEI/XhoI fragment containing Fc with a G10 hinge were then subcloned into pDSRα19. This plasmid produces OPG[22-194]-FcG10 upon expression in mammalian cells. The amino acid sequence is shown in FIG. 7.

| | |
|---|---|
| G:10-Fc 5′ Primer: | |
| | BspEI Gly Linker KpnI Fc Domain → |
| 1775-30 | 5′-AA TCCGGA GGAGGTGGTGGAGGTGGG GGTACC TGCCCACCGTGC-3′ |
| | S G G G G G G G G T C P P C |
| G10-Fc 3′ Primer: | |
| | XhoI |
| 1504-63 | 5′-CCATG CTCGAG TTA TCA TTT ACC CGG AGA CAG G-3′ |
| | * * K G P S L |
| OPG 5′ Primer: | |
| | XbaI Kozak opg coding→ |
| 1745-92 | 5′-AAG TCTAGA CCACC ATG AAC AAG TTG CTG T-3′ |
| | M N K L L |
| OPG 3′ Primer: | |
| | BspEI |
| 1790-72 | 5′- CC TCCGGA TTT TTG AGT TGA TTC ACT GTT TCC AGA-3′ |
| | K Q T S E S N G S |

Construction of FcΔC-OPG[22-194]

A DNA molecule encoding FcΔC-OPG [22-194] was generated by standard PCR techniques using the pDSRα2:OPG[1-201]-Fc DNA as a template. The Fc portion was generated using oligonucleotides 1757-22 and 1757-23. The 1757-22 primer has an in frame Epo BssHII signal to place the Fc downstream from the erythropoeitin signal sequence (the signal sequence is described in U.S. Pat. No. 4,703,008). The 1757-23 primer fuses the last amino acid of the Fc domain to amino acid residue 22 of human OPG. The OPG portion was generated using oligonucleotides 1757-24 and 1789-04. The 1789-04 primer places a stop codon after amino acid 194 of human OPG and a SalI site for cloning. These two purified products were then used as a template to generate the Fc/OPG fusion molecule with primers 1757-22 and 1789-04. The resulting PCR product was digested with BssHII and SalI, purified and cloned into BssHII/SalI digested pDSRα19. Expression of this plasmid in a mammalian host cell produces FcΔC-OPG[22-194] as shown in FIG. 8 (SEQ ID NO: ______) with the modification that the amino terminal methionine is replaced with the amino acids ala-pro.

| | |
|---|---|
| Sense Fc primer (1757-22): | 5′-TTG GCGCGC CCA AAT CTT GTG ACA AAA CT-3′ |
| | BssNII |
| Antisense Fc OPG primer (1757-23): | 5′-CTT TGG AGG AAA CGT TTC TTT ACC CGG AGA CAG GGA-3′ |
| | OPG →\| ←Fc |
| Sense Fc OPG Primer (1757-24): | 5′-TCC CTG TCT CCG GGT AAA GAA ACG TTT CCT CCA AAG-3′ |
| | Fc →\| ←OPG |
| Antisense OPG Primer (1789-04): | 5′-ATCT GTCGA CTA TTT TTG AGT TGA TTC AC-3′ |
| | SalI * OPG Coding |

The vector pDSRα2 has been described previously (see WO90/14363 and FIG. 12 therein, which is incorporated by reference). The vector pDSRα19 is a modified form of pDSRα2 which is functionally similar but contains the following changes from pDSRα2:

1) The αFSH polyA was shortened approximately 1400 bp from the 3′ end. It is now 885 bp and ends at the NdeI site.

2) The dihydrofolate reductase (DHFR) promoter was shortened from the 5′ end by approximately 1 kb and now only contains 209 bp.

3) An approximately 550 bp BglII fragment in the DHFR polyA was deleted.

Conditions for the purification of truncated and fusion polypeptides from conditioned media are generally described in WO97/23614

Construction of met FcΔc-OPG[22-194]

A met huOPG[22-194] coding sequence was constructed by the following procedure. Synthetic oligonucleotides were constructed consisting of overlapping 50-mers which comprised the entire top and bottom strands of the OPG DNA coding sequence. The internal 50-mer oligos were phosphorylated, annealed, and ligated overnight. The outside oligos, 34-mers, were used in the polymerase chain reaction (PCR) as primers to amplify the full length gene. The PCR reaction was performed using Taq DNA polymerase and additional reaction components as supplied in kit form (Boehringer Mannheim). The resulting 584 base pair PCR product was purified by 1% agarose gel electrophoresis and extracted from the gel using the QIAquick spin column method (Qiagen). The gel purified fragment was then digested with the restriction enzymes XbaI and BamHI (Boehringer Mannheim). A ligation reaction was performed with the fragment described above and the plasmid vector pAMG21 (ATCC accession number 98113) digested with the same restriction enzymes. The ligated DNA was transformed by electroporation into E. coli strain #393. Clones were selected for kanamycin antibiotic resistance, plasmid was isolated, and the sequence of the coding region was checked by DNA sequencing. The initial clone selected (referred to as plasmid A) was shown by DNA sequencing to have significant errors in the middle of the gene. The gene sequence was repaired by digesting plasmid A with the restriction enzymes SpeI and HpaI and using the resulting product as the vector fragment. A new insert fragment was prepared by PCR of the original ligated oligonucleotide mixture with internal oligonucleotides 1466-91 and 1467-03 as PCR primers in the polymerase chain reaction. The insert fragment was digested with SpeI and HpaI and ligated into the plasmid A vector to replace the DNA fragment containing the errors. Transformation, selection, and plasmid isolation were performed as described above. A clone (plasmid B) was confirmed by DNA sequencing as having the correct sequence for human OPG[22-194].

Top strand oligonucleotides 1466-90 to 1467-01:

| | |
|---|---|
| 1466-90 | |
| 5′AACAAACTCTAGATTTGTTTTAACTAATTAAAGG-3′ | (SEQ ID NO:___) |
| 1466-91 | |
| 5′AGGAATAACATATGGAAACTTTTCCACCTAAATATCTTCATTATGATGAA-3′ | (SEQ ID NO:___) |
| 1466-02 | |
| 5′GAAACTAGTCACCAGCTGCTGTGCGACAAATGTCCTCCGGGTACCTACCT-3′ | (SEQ ID NO:___) |
| 1466-93 | |
| 5′GAAACAGCACTGCACCGCTAAATGGAAAACCGTTTGCGCTCCTTGTCCGG-3′ | (SEQ ID NO:___) |
| 1466-94 | |
| 5′ACCACTACTACACCGACTCCTGGCACACCTCCGACGAATGCCTGTACTGC-3′ | (SEQ ID NO:___) |
| 1466-95 | |
| 5′TCACCGGTTTGCAAGGAGCTGCAGTACGTTAAACAGGAATGCAACCGTAC-3′ | (SEQ ID NO:___) |
| 1466-96 | |
| 5′GCACAACCGTGTTTGCGAATGCAAAGAAGGTCGTTACCTGGAGATCGAAT-3′ | (SEQ ID NO:___) |
| 1466-97 | |
| 5′TCTGCCTGAAACACCGTTCCTGTCCGCCTGGTTTCGGTGTTGTACAGGCT-3′ | (SEQ ID NO:___) |
| 1466-98 | |
| 5′GGTACCCCGGAACGTAACACCGTTTGCAAACGTTGCCCGGACGGTTTCTT-3′ | (SEQ ID NO:___) |
| 1466-99 | |
| 5′CTCCAACGAAACCTCGAGCAAAGCTCCGTGCCGTAAACACACCAACTGCT-3′ | (SEQ ID NO:___) |
| 1466-00 | |
| 5′CCGTTTTCGGTCTCCTGTTAACCCAGAAAGGTAACGCTACCCACGACAAC-3′ | (SEQ ID NO:___) |
| 1466-01 | |
| 5′ATCTGCTCCGGTAACTCCGAGTCGACCCAGAAATAATGGATCCCAAACAA-3′ | (SEQ ID NO:___) |
| Bottom strand oligonucleotides 1476-02 through 1476-13 | |
| 1466-02 | |
| 5′-TTGTTTGGGATCCATTATTTCTGGGTCGACTCGG-3′ | (SEQ ID NO:___) |
| 1466-03 | |
| 5′AGTTACCGGAGCAGATGTTGTCGTGGGTAGCGTTACCTTTCTGGGTTAAC-3′ | (SEQ ID NO:___) |
| 1466-04 | |
| 5′AGGAGACCGAAAACGGAGCAGTTGGTGTGTTTACGGCACGGAGCTTTGCT-3′ | (SEQ ID NO:___) |
| 1466-05 | |
| 5′CGAGGTTTCGTTGGAGAAGAAACCGTCCGGGCAACGTTTGCAAACGGTGT-3′ | (SEQ ID NO:___) |
| 1466-06 | |
| 5′TACGTTCCGGGGTACCAGCCTGTACAACACCGAAACCAGGCGGACAGGAA-3′ | (SEQ ID NO:___) |
| 1466-07 | |
| 5′CGGTGTTTCAGGCAGAATTCGATCTCCAGGTAACGACCTTCTTTGCATTC-3′ | (SEQ ID NO:___) |
| 1466-08 | |
| 5′GCAAACACGGTTGTGCGTACGGTTGCATTCCTGTTTAACGTACTGCAGCT-3′ | (SEQ ID NO:___) |
| 1466-09 | |
| 5′CCTTGCAAACCGGTGAGCAGTACAGGCATTCGTCGGAGGTGTGCCAGGAG-3′ | (SEQ ID NO:___) |
| 1466-10 | |
| 5′TCGGTGTAGTAGTGGTCCGGACAAGGAGCGCAAACGGTTTTCCATTTAGC-3′ | (SEQ ID NO:___) |
| 1466-11 | |
| 5′GGTGCAGTGCTGTTTCAGGTAGGTACCCGGAGGACATTTGTCGCACAGCA-3′ | (SEQ ID NO:___) |
| 1466-12 | |
| 5′GCTGGTGACTAGTTTCTTCATCATAATGAAGATATTTAGGTGGAAAAGTT-3′ | (SEQ ID NO:___) |
| 1467-13 | |
| 5′TCCATATGTTATTCCTCCTTTAATTAGTTAAAACAAATCTAGAGTTTGTT-3′ | (SEQ ID NO:___) |

Fusion of human OPG[22-194] DNA sequence described above to human IgG₁ FcΔC was performed as follows. Plasmid DNA comprising an insert of OPG DNA coding sequence described above fused at its amino terminus to the Fc region of plasmid pFc-A3 was digested with the restriction enzymes NdeI and SpeI. Plasmid pFc-A3 has been described in WO97/23614. The resulting plasmid vector fragment contained the OPG coding sequence minus the first fourteen codons of the gene (up to the SpeI site). This was designated as vector C. The insert was created by performing the polymerase chain reaction using a DNA sequence as shown in SEQ ID NO:13 and SEQ ID NO:14 as set forth in WO98/28427 as the template. A universal 5′ primer (#1209-85) for the plasmid pAMG21 (ATCC accession no. 98113) was used to prime the 5′ end of the Fc sequence (an NdeI site already existed at the beginning of the Fc sequence). Two oligonucleotide primers were designed to prime at the 3′ end of the Fc coding sequence while adding an overlap region identical to the 5′ end of the osteoprotegerin gene. The first primer, 1595-18, was designed to prime the 3′ end of the Fc coding sequence and add the first codons of the 5′ end of the osteoprotegerin sequence. A second primer, 1585-16, primed at the 3′ end of the previously mentioned primer and added additional OPG coding sequence through the SpeI site at codon fourteen. The first round of PCR was performed using a DNA molecule having the sequence in SEQ ID NO:13 and SEQ ID NO:14 of WO98/28427 as template, and primers 1209-85 and 1595-18 with Taq polymerase as previously described. The 799 base pair PCR product of this reaction was gel purified and used as template in a second PCR reaction with primers 1209-85 and 1585-16. The 825 base pair product of the second PCR reaction was gel purified, digested with NdeI and SpeI, and ligated into vector C described above. The ligation mixture was transformed into E. coli and a clone was isolated and confirmed by DNA sequencing to have the correct OPG coding sequence. The resulting plasmid encodes [met]FcΔC-huOPG [22-194] having the amino acid sequence shown in FIG. 8 (SEQ ID NO:______).

| | |
|---|---|
| Primer 1209-85: | |
| 5′-CGTACAGGTTTACGCAAGAAAATGG-3′ | (SEQ ID NO:___) |
| Primer 1585-16: | |
| 5′ACAAACACTAGTTTCTTCATCATAATGAAGATATTTAGGTGGAAACGT-3′ | (SEQ ID NO:___) |
| Primer 1595-18: | |
| 5′GAAGATATTTAGGTGGAAACGTTTCTTTACCCGGAGACAGGGAG-3′ | (SEQ ID NO:___) |

Expression of a DNA sequence encoding [met]FcdC-huOPG[22-194] in pAMG21 was performed generally as described in WO97/23614. The fusion polypeptide was purified by conventional procedures.

### EXAMPLE 2

### Activity of OPG Polypeptides

The in vivo activity of selected OPG polypeptides and OPG fusion polypeptides described in Example 1 was determined as follows. OPG preparations were administered by subcutaneous (SC) injection to 4-5 week old male BDF1 mice for 4 days and radiographs of the mice were taken on day 5. The positive result was for increased radiographic density in the proximal tibial metaphysis compared to vehicle treated controls. There were 4 animals per group with each tibia compared to a different control tibia to give the results numbered 1-8. At least 5 of 8 results were required to be positive in order to conclude that a biological response had occurred. The lowest dose giving a biological response is considered the indicator of in vivo potency. All doses are expressed as mg/kg/day. Daily dose experiments with truncated and full-length OPG polypeptides are shown in Table 2. Daily dose experiments with OPG fusion polypeptides are shown in Table 3. OPG polypeptides and OPG fusion polypeptides having an N-terminal methionine residue were expressed in E. coli host cells, while those without an N-terminal methionine were expressed in CHO cells.

TABLE 2
Daily Dosing Experiments
X ray on day 5

| Factor | Dose | 1 | 2 | 3 | 4 | 5 | 6 | 7 | 8 | Results |
|---|---|---|---|---|---|---|---|---|---|---|
| met OPG[22-194] | 10.0 | − | − | + | + | + | + | + | − | Positive 5/8 |
| met OPG[22-194] | 5.0 | − | − | − | − | − | + | − | − | Negative 1/8 |
| met OPG[22-194] | 1.0 | − | − | − | − | − | − | − | − | Negative 0/8 |
| met OPG[22-201] | 1.5 | − | + | − | + | + | + | + | + | Positive 6/8 |
| met OPG[22-201] | 0.5 | − | − | − | + | − | − | − | + | Negative 2/8 |
| met OPG[22-201] | 0.15 | − | − | − | − | − | + | − | − | Negative 1/8 |
| OPG[22-293] | 1.5 | + | + | + | + | + | + | + | + | Positive 8/8 |
| OPG[22-293] | 0.5 | + | + | + | + | + | + | + | + | Positive 8/8 |
| OPG[22-293] | 0.15 | − | − | − | − | − | − | − | − | Negative 0/8 |
| OPG[22-401] | 10 | − | + | + | + | + | + | − | − | Positive 5/8 |
| OPG[22-401] | 4.2 | − | − | − | − | − | + | + | − | Negative 2/8 |

TABLE 3
Daily Dosing Experiments
X ray on day 5

| Factor | Dose | 1 | 2 | 3 | 4 | 5 | 6 | 7 | 8 | Result |
|---|---|---|---|---|---|---|---|---|---|---|
| met FcΔC-22-194 | 0.05 | + | + | + | + | + | + | + | + | Positive 8/8 |
| met FcΔC-22-194 | 0.015 | − | − | + | − | + | + | − | − | Negative 3/8 |
| met BcΔC-22-194 | 0.005 | − | − | − | − | − | − | − | − | Negative 0/8 |
| FcΔC-OPG[22-194] | 0.15 | + | + | + | + | + | + | + | + | Positive 8/8 |
| FcΔC-OPG[22-194] | 0.05 | + | − | − | − | + | + | + | + | Positive 5/8 |
| FcΔC OPG[22-194] | 0.015 | − | − | + | − | + | − | + | − | Negative 3/8 |
| OPG[22-201]-Fc | 0.05 | + | + | + | + | + | + | + | + | Positive 8/8 |
| OPG[22-201]-Pc | 0.015 | − | + | − | − | + | + | + | + | Positive 5/8 |
| OPG[22-201]-Fc | 0.005 | − | − | − | − | − | − | − | − | Negative 0/8 |
| OPG[22-194]-FcΔC | 0.05 | + | + | + | + | + | + | + | + | Positive 8/8 |
| OPG[22-194]-FcΔC | 0.015 | − | + | − | + | + | − | + | + | Positive 5/8 |
| OPG[22-194]-FcΔC | 0.005 | − | − | − | − | − | − | − | − | Negative 0/8 |

In single dose experiments, male BDF1 mice aged 3-4 weeks received varying doses of OPG fusion proteins indicated below by a single subcutaneous injection in carrier (PBS/0.1% BSA) on day 0 (or day 1), the mice were then x-rayed on day 7 (or day 5). For each treatment, all the mice in that group and the PBS/0.1% BSA control group were x-rayed on a single film. Positive results were scored as describe above.

Doses are expressed in mg/Kg. The results are shown in Table 4.

TABLE 4
Single Dose Experiments
X ray on day 5

| Factor | Dose | 1 | 2 | 3 | 4 | 5 | 6 | 7 | 8 | Results |
|---|---|---|---|---|---|---|---|---|---|---|
| met FcΔC-22-194 | 0.3 | + | + | + | + | + | + | + | + | Positive 8/8 |
| met gcΔC-22-194 | 0.1 | − | + | − | − | − | − | + | + | Negative 3/8 |
| met FcΔC-22-194 | 0.03 | − | − | − | − | − | − | − | − | Negative 0/8 |
| FCΔC-OPG[22-194] | 0.3 | + | + | + | + | − | + | + | + | Positive 7/8 |
| FcΔC-OPG[22-194] | 0.1 | − | − | − | − | − | − | − | − | Negative 0/8 |
| FcΔC-OPG[22-194] | 0.03 | − | − | − | − | − | − | − | − | Negative 0/8 |
| OPG[22-201]-Fc | 0.3 | + | + | + | + | + | + | + | + | Positive 8/8 |
| OPG[22-201]-Fc | 0.1 | + | + | + | + | + | + | + | + | Positive 8/8 |
| OPG[22-201]-Fc | 0.03 | + | − | + | + | − | − | − | − | Negative 3/8 |
| OPG[22-194]-FcΔC | 0.3 | + | + | + | + | + | + | + | + | Positive 8/8 |
| OPG[22-194]-FcΔC | 0.1 | + | + | + | + | + | + | + | + | Positive 8/8 |
| OPG[22-194]-FcΔC | 0.03 | − | − | + | + | − | − | + | + | Negative 4/8 |

Single Dose Experiments
X ray on day 7

| | | | | | | | | | | |
|---|---|---|---|---|---|---|---|---|---|---|
| met FcΔC-22-194 | 3.0 | + | + | + | + | + | + | + | + | Positive 8/8 |
| met FcΔC-22-194 | 1.0 | − | − | + | + | + | − | + | + | Positive 5/8 |
| met FcΔC-22-194 | 0.3 | − | − | − | − | − | − | − | − | Negative 0/8 |
| met FcΔC-22-194 | 0.1 | − | − | − | − | − | − | − | − | Negative 0/8 |
| OPG[22-194]-FcΔC | 3.0 | + | + | + | + | + | + | + | + | Positive 8/8 |
| OPG[22-194]-FcΔC | 1.0 | + | + | + | + | + | + | + | + | Positive 8/8 |
| OPG[22-194]-FcΔC | 0.3 | + | + | + | + | + | + | + | + | Positive 8/8 |
| OPG[22-194]-FcΔC | 0.1 | − | + | − | − | + | − | + | − | Negative 3/8 |

It is apparent that OPG truncated polypeptides fused to an Fc region demonstrate in vivo activity at lower doses than unfused OPG truncated or full-length polypeptides. Further, OPG[22-194]-FcΔC (Fc fusion at the carboxy terminus of OPG[22-194] polypeptide) demonstrated greater in vivo potency than FcΔC-OPG[22-194](Fc fusion at the amino terminus of OPG[22-194]).

While the present invention has been described in terms of preferred embodiments, it is understood that variations and modifications will occur to those skilled in the art. Therefore, it is intended that the appended claims cover all such equivalent variations which come within the scope of the invention as claimed.

What is claimed is:

1. A protein having a formula selected from the group consisting of: R₁-R₂, R₂-R₁, R₁-L-R₂ and R₂-L-R₁, wherein R₁ is a Fc protein, or variant or fragment thereof, R₂ is an OPG protein, or variant or fragment thereof, and L is a linker.

2. The protein of claim 1 having the formula R₂-L-R₁.

3. The protein according to claim 1, wherein the Fc protein is selected from the group consisting of:
(a) the Fc amino acid sequences as set forth in FIG. 1;
(b) the amino acid sequence of subpart (a) having a different amino acid substituted or deleted in one or more of the following positions (using the numbering according to FIG. 1):
(i) one or more cysteine residues;
(ii) one or more tyrosine residues;
(iii) cysteine at position 5 deleted or substituted with an alanine;
(iv) leucine at position 20 deleted or substituted with glutamine;
(v) glutamic acid at position 103 deleted or substituted with an alanine;
(vi) lysine at position 105 deleted or substituted with an alanine;
(vii) lysine at position 107 deleted or substituted with an alanine;
(viii) deletion or substitution of one or more of the amino acids at positions 1, 2, 3, 4, and 5;
(ix) one or more residues substituted or deleted to ablate the Fc receptor binding site;
(x) one or more residues substituted or deleted to ablate the complement (C1q) binding site; and
(xi) a combination of subparts i-x;
(c) the amino acid sequence of subparts (a) or (b) having a methionyl residue at the N-terminus;
(d) the Fc protein, or variant, fragment or derivative thereof, of any of subparts (a) through (c) comprised of a chemical moiety connected to the protein moiety;
(e) a derivative of subpart (d) wherein said chemical moiety is a water soluble polymer moiety;
(f) a derivative of subpart (e) wherein said water soluble polymer moiety is polyethylene glycol; and
(g) a derivative of subpart (e) wherein said water soluble polymer moiety is attached at solely the N-terminus of said protein moiety.

4. The protein according to claim 1, wherein the OPG protein, or variant, fragment or derivative thereof, is selected from the group consisting of:
(a) the amino acid sequence 22-X wherein X is any residue from position 185 to 401 inclusive as shown in FIG. 2 (SEQ ID NO:______);
(b) the amino acid sequence 22-X wherein X is any residue from position 185 to 293 inclusive as shown in FIG. 2 (SEQ ID NO:______);
(c) the amino acid sequence of subparts (a) and (b) having a methionyl residue at the N-terminus.
(c) the OPG protein, or variant, fragment or derivative thereof, of any of subparts (a),(b) and (c) comprised of a chemical moiety connected to the protein moiety;
(d) a derivative of subpart (c) wherein said chemical moiety is a water soluble polymer moiety;
(e) a derivative of subpart (d) wherein said water soluble polymer moiety is polyethylene glycol;
(f) A derivative of subpart (d) wherein said water soluble polymer moiety is a polyamino acid moiety; and
(g) a derivative of subpart (d) wherein said water soluble polymer moiety is attached at solely the N-terminus of said protein moiety.

5. The protein of claim 1 wherein the linker is one or more amino acids selected from the group consisting of glycine, asparagine, serine, threonine and alanine.

6. The protein of claim 1 wherein the linker is selected from the group consisting of:

| | |
|---|---|
| (a) | ala-ala-ala; |
| (b) | ala-ala-ala-ala; |
| (c) | ala-ala-ala-ala-ala; |
| (d) | gly-gly; |
| (e) | gly-gly-gly; |
| (f) | gly-gly-gly-gly-gly; |
| (g) | gly-gly-gly-gly-gly-gly-gly; |
| (h) | gly-pro-gly; |
| (i) | gly-gly-pro-gly-gly; |
| (j) | val; |
| (k) | ser-gly-gly-gly-gly-gly-gly-gly-gly; |
| (l) | gly-gly-ser-gly-ser-ala-gly-ser-gly-ser-gly-gly-gly-ser-gly-ser-gly-gly; |
| (m) | a chemical moiety; and |
| (n) | any combination of subparts (a) |
| | through (m). |

7. A fusion protein comprising the amino acid sequence selected from the group consisting of the amino acid sequences set forth in FIGS. 5, 6, 7 or 8 (SEQ ID NOS: ______, ______, ______, ______, respectively).

8. A nucleic acid sequence encoding for a protein having the formula selected from the group consisting of: R₁-R₂, R₂-R₁, R₁-L-R₂, and R₂-L-R₁ wherein R₁ is a Fc protein, or variant or fragment thereof, R₂ is an OPG protein, or variant or fragment thereof, and L is a linker.

9. The nucleic acid sequence of claim 8 encoding for a protein comprising an Fc protein, variant, fragment or derivative portion selected from the group consisting of:
(a) the Fc amino acid sequence as set forth in FIG. 1 (SEQ ID NO:______);
(b) the amino acid sequence of subpart (a) having a different amino acid substituted or deleted in one or more of the following positions (using the numbering according to FIG. 1):
(i) one or more cysteine residues;
(ii) one or more tyrosine residues;
(iii) cysteine at position 5 deleted or substituted with an alanine;
(iv) leucine at position 20 deleted or substituted with glutamine;
(v) glutamic acid at position 103 deleted or substituted with an alanine;
(vi) lysine at position 105 deleted or substituted with an alanine;
(vii) lysine at position 107 deleted or substituted with an alanine;
(viii) deletion or substitution of one or more of the amino acids at positions 1, 2, 3, 4, and 5;
(ix) one or more residues substituted or deleted to ablate the Fc receptor binding site;
(x) one or more residues substituted or deleted to ablate the complement (C1q) binding site; and
(xi) a combination of subparts i-x;
(c) the amino acid sequence of subparts (a) or (b) having a methionyl residue at the N-terminus;
(d) the Fc protein, or variant, fragment or derivative thereof, of any of subparts (a) through (c) comprised of a chemical moiety connected to the protein moiety;
(e) a derivative of subpart (d) wherein said chemical moiety is a water soluble polymer moiety;
(f) a derivative of subpart (e) wherein said water soluble polymer moiety is polyethylene glycol; and
(g) a derivative of subpart (e) wherein said water soluble polymer moiety is attached at solely the N-terminus of said protein moiety.

10. The nucleic acid sequence according to claim 8 encoding for a protein comprising an OPG protein, variant, fragment or derivative portion selected from the group consisting of:
(a) the amino acid sequence 22-X wherein X is any residue from position 185 to 401 inclusive as shown in FIG. 2 (SEQ ID NO:______);
(b) the amino acid sequence 22-X wherein X is any residue from position 185 to 293 inclusive as shown in FIG. 2 (SEQ ID NO:______);
(c) the amino acid sequence of subparts (a) and (b) having a methionyl residue at the N-terminus;
(d) the OPG protein, or variant, fragment or derivative thereof, of any of subparts (a),(b) and (c) comprised of a chemical moiety connected to the protein moiety;
(e) a derivative of subpart (d) wherein said chemical moiety is a water soluble polymer moiety;
(f) a derivative of subpart (e) wherein said water soluble polymer moiety is polyethylene glycol;
(g) A derivative of subpart (e) wherein said water soluble polymer moiety is a polyamino acid moiety; and
(h) a derivative of subpart (e) wherein said water soluble polymer moiety is attached at solely the N-terminus of said protein moiety.

11. The nucleic acid sequence of claim 8 encoding for a protein comprising a linker of one or more amino acids selected from the group consisting of glycine, asparagine, serine, threonine and alanine.

12. The nucleic acid sequence of claim 8 encoding for a protein with a linker selected from the group consisting of:

| | |
|---|---|
| (a) | ala-ala-ala; |
| (b) | ala-ala-ala-ala; |
| (c) | ala-ala-ala-ala-ala; |
| (d) | gly-gly; |
| (e) | gly-gly-gly; |
| (f) | gly-gly-gly-gly-gly; |
| (g) | gly-gly-gly-gly-gly-gly-gly; |
| (h) | gly-pro-gly; |
| (i) | gly-gly-pro-gly-gly; |
| (j) | val; |
| (k) | ser-gly-gly-gly-gly-gly-gly-gly-gly; |
| (l) | gly-gly-ser-gly-ser-gly-ala-gly- |
| | ser-gly-ser-gly-gly-gly-ser-gly-ser-gly-gly; |
| (m) | a chemical moiety; and |
| (n) | any combination of subparts (a) through (m). |

13. A nucleic acid sequence encoding a fusion protein comprising the amino acid sequence selecting from the group consisting of: the amino acid sequences as set forth in FIGS. 5, 6, 7 or 8 (SEQ ID NOS:______, ______, ______, ______, respectively).

14. A vector comprising a nucleic acid sequence according to any of claims 8 to 13 inclusive.

15. A prokaryotic or eukaryotic host cell containing the vector of claim 14.

16. A process for producing a protein of claims 1 or 6 comprising the steps of culturing, under suitable conditions, the host cell of claim 15, and isolating the protein produced.

17. The process of claim 16 further comprising the step of purifying the protein produced.

18. A pharmaceutical composition comprising an effective amount of a protein according to claims 1 or 6, in a pharmaceutically acceptable diluent, adjuvant or carrier.

19. A method of preventing or treating a bone loss in a mammal comprising administering a therapeutically effective amount of the protein of any of claims 1-6.

20. The method of claim 19 wherein the bone Loss is selected from the group consisting of osteoporosis, Paget's disease, osteomyelitis, hypercalcemia, osteopenia associated with surgery or steroid administration, osteonecrosis, bone loss due to rheumatoid arthritis, periodontal bone loss, osteolytic metastasis, and prosthetic loosening.
